SEQUENCE LISTING (10 sequences)

SEQ ID NO: 1 - protein, 1444 aa, Plasmodium falciparum
MKLFDSFFKHALIRINKRNIIYLNATRYYCNNINYNALINLLNKKNDINKEINALYSLLERLSNYKYKQYKDKLTLKNNINDEIKITNADKINNINIERDMNISHLDHHHNNHHHNNNHHNINHNNHHHNNHHNINHHNNHHNNHHNNNHFNDYKKLIDNWKNDKIKIFISWCPEIVEDKYKSKCFSIPTYITFHIVITNNDIKLNNLLHNSHEYDDWNFNKIIQTINNQNNLKDKEKEKENGQQHSQEYIGNCKKGESEIPSYDFKESLLEHINESSQLNHSILSHKTKEQTHHTNNNINGNYNNDEHIEEEGKAKTKQNKTKNSIIEEKKKKTKKKKDEESHNDIINYTIKKKTNTNNSLYNIESILNIPKTYEPNIHYDKCIHKEQNHIFFFSFNISELIINDQVKTKLNECIQQNFIKQNISNIHMDDLFLYVVYDYKNLIHIFNNINLKLININNIFDIYIISSLIQLVQRGEKLQNVYNEYLNVKHKILIPNKINDIQNLSLHNFSYFSKFAPEFSDVISAKFGLYGWGKYQKKKDKKNKKQTENHENNENYENNEYGKNNEYGKNNEYGKNNEYGKNNEYGKNNEYGKNNEYGKNNEYGKNNVHNDDTYMDISNERKNKKSKEVKNKKKMEKKNKVEKEKQNYLSFTPHNINNLQDIKKLVFGNKRNISDITEEDNICYSISRNCCLILLFEYFINKLEHNINILNLYIKVEQPLILCISHIEEKGIFLNQNKIEEIQKKSDDPLIYKNEIEELCKCNINLNSSKQVSSLIYKQLLDISISTDHTEENMEDADEHTDHQEEEHVNDDNNECVDQLKAYTQTKEKERKDIYNNNNNENNKNNNENYNSSKNHPLITNTNNDDTSTLNAQDTSDQHDNYINEHNNYNKFIKNNPFYYNNNNNNNNNNNNNNDNNNNISNRNLMNNLVNINYTSLYNKKKNSHPYDENNKLFFLNSSHNNYNNNNNNINEMSRNKNLQTNNKSLKILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 2 - protein, 1341 aa, Plasmodium falciparum
SHLDHHHNNHHHNNNHHNINHNNHHHNNHHNINHHNNHHNNHHNNNHFNDYKKLIDNWKNDKIKIFISWCPEIVEDKYKSKCFSIPTYITFHIVITNNDIKLNNLLHNSHEYDDWNFNKIIQTINNQNNLKDKEKEKENGQQHSQEYIGNCKKGESEIPSYDFKESLLEHINESSQLNHSILSHKTKEQTHHTNNNINGNYNNDEHIEEEGKAKTKQNKTKNSIIEEKKKKTKKKKDEESHNDIINYTIKKKTNTNNSLYNIESILNIPKTYEPNIHYDKCIHKEQNHIFFFSFNISELIINDQVKTKLNECIQQNFIKQNISNIHMDDLFLYVVYDYKNLIHIFNNINLKLININNIFDIYIISSLIQLVQRGEKLQNVYNEYLNVKHKILIPNKINDIQNLSLHNFSYFSKFAPEFSDVISAKFGLYGWGKYQKKKDKKNKKQTENHENNENYENNEYGKNNEYGKNNEYGKNNEYGKNNEYGKNNEYGKNNEYGKNNEYGKNNVHNDDTYMDISNERKNKKSKEVKNKKKMEKKNKVEKEKQNYLSFTPHNINNLQDIKKLVFGNKRNISDITEEDNICYSISRNCCLILLFEYFINKLEHNINILNLYIKVEQPLILCISHIEEKGIFLNQNKIEEIQKKSDDPLIYKNEIEELCKCNINLNSSKQVSSLIYKQLLDISISTDHTEENMEDADEHTDHQEEEHVNDDNNECVDQLKAYTQTKEKERKDIYNNNNNENNKNNNENYNSSKNHPLITNTNNDDTSTLNAQDTSDQHDNYINEHNNYNKFIKNNPFYYNNNNNNNNNNNNNNDNNNNISNRNLMNNLVNINYTSLYNKKKNSHPYDENNKLFFLNSSHNNYNNNNNNINEMSRNKNLQTNNKSLKILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 3 - protein, 1169 aa, Plasmodium falciparum
ESSQLNHSILSHKTKEQTHHTNNNINGNYNNDEHIEEEGKAKTKQNKTKNSIIEEKKKKTKKKKDEESHNDIINYTIKKKTNTNNSLYNIESILNIPKTYEPNIHYDKCIHKEQNHIFFFSFNISELIINDQVKTKLNECIQQNFIKQNISNIHMDDLFLYVVYDYKNLIHIFNNINLKLININNIFDIYIISSLIQLVQRGEKLQNVYNEYLNVKHKILIPNKINDIQNLSLHNFSYFSKFAPEFSDVISAKFGLYGWGKYQKKKDKKNKKQTENHENNENYENNEYGKNNEYGKNNEYGKNNEYGKNNEYGKNNEYGKNNEYGKNNEYGKNNVHNDDTYMDISNERKNKKSKEVKNKKKMEKKNKVEKEKQNYLSFTPHNINNLQDIKKLVFGNKRNISDITEEDNICYSISRNCCLILLFEYFINKLEHNINILNLYIKVEQPLILCISHIEEKGIFLNQNKIEEIQKKSDDPLIYKNEIEELCKCNINLNSSKQVSSLIYKQLLDISISTDHTEENMEDADEHTDHQEEEHVNDDNNECVDQLKAYTQTKEKERKDIYNNNNNENNKNNNENYNSSKNHPLITNTNNDDTSTLNAQDTSDQHDNYINEHNNYNKFIKNNPFYYNNNNNNNNNNNNNNDNNNNISNRNLMNNLVNINYTSLYNKKKNSHPYDENNKLFFLNSSHNNYNNNNNNINEMSRNKNLQTNNKSLKILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 4 - protein, 1019 aa, Plasmodium falciparum
SNIHMDDLFLYVVYDYKNLIHIFNNINLKLININNIFDIYIISSLIQLVQRGEKLQNVYNEYLNVKHKILIPNKINDIQNLSLHNFSYFSKFAPEFSDVISAKFGLYGWGKYQKKKDKKNKKQTENHENNENYENNEYGKNNEYGKNNEYGKNNEYGKNNEYGKNNEYGKNNEYGKNNEYGKNNVHNDDTYMDISNERKNKKSKEVKNKKKMEKKNKVEKEKQNYLSFTPHNINNLQDIKKLVFGNKRNISDITEEDNICYSISRNCCLILLFEYFINKLEHNINILNLYIKVEQPLILCISHIEEKGIFLNQNKIEEIQKKSDDPLIYKNEIEELCKCNINLNSSKQVSSLIYKQLLDISISTDHTEENMEDADEHTDHQEEEHVNDDNNECVDQLKAYTQTKEKERKDIYNNNNNENNKNNNENYNSSKNHPLITNTNNDDTSTLNAQDTSDQHDNYINEHNNYNKFIKNNPFYYNNNNNNNNNNNNNNDNNNNISNRNLMNNLVNINYTSLYNKKKNSHPYDENNKLFFLNSSHNNYNNNNNNINEMSRNKNLQTNNKSLKILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 5 - protein, 827 aa, Plasmodium falciparum
DISNERKNKKSKEVKNKKKMEKKNKVEKEKQNYLSFTPHNINNLQDIKKLVFGNKRNISDITEEDNICYSISRNCCLILLFEYFINKLEHNINILNLYIKVEQPLILCISHIEEKGIFLNQNKIEEIQKKSDDPLIYKNEIEELCKCNINLNSSKQVSSLIYKQLLDISISTDHTEENMEDADEHTDHQEEEHVNDDNNECVDQLKAYTQTKEKERKDIYNNNNNENNKNNNENYNSSKNHPLITNTNNDDTSTLNAQDTSDQHDNYINEHNNYNKFIKNNPFYYNNNNNNNNNNNNNNDNNNNISNRNLMNNLVNINYTSLYNKKKNSHPYDENNKLFFLNSSHNNYNNNNNNINEMSRNKNLQTNNKSLKILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 6 - protein, 713 aa, Plasmodium falciparum
KGIFLNQNKIEEIQKKSDDPLIYKNEIEELCKCNINLNSSKQVSSLIYKQLLDISISTDHTEENMEDADEHTDHQEEEHVNDDNNECVDQLKAYTQTKEKERKDIYNNNNNENNKNNNENYNSSKNHPLITNTNNDDTSTLNAQDTSDQHDNYINEHNNYNKFIKNNPFYYNNNNNNNNNNNNNNDNNNNISNRNLMNNLVNINYTSLYNKKKNSHPYDENNKLFFLNSSHNNYNNNNNNINEMSRNKNLQTNNKSLKILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 7 - protein, 455 aa, Plasmodium falciparum
ILVDEIEKSNYIKEKEKEKLKKIIRNIKLYRESKKLVQNYIENLPKYIQKNTNKIHCNFNQIGASTGRLSCDQPNLQNIHSRFRCAISLKGKEENDTHDNNNNNNNIPQIHISTNNVSTNNVPMNIMSSTYPLYTMNKKNLITFDYKQMELFVMAYLSFDEQLLKLLNYSDVFIETAKVLFNTNDVTNELRRMTKTVIYGILYGQTENGLAKSLLISDTLASNLIENFFQFFPNVYRFMQMQKFLVKHMNCVYTLIGRKRIILPNIKNKYRISMNTPIQGCAADIMKFSLLSCFSVLNNNIYNNNKLLKMNNINPLIIHKNQAFLNPTNLILQVHDELLLESEHDATKYIIQLLNPILENAFYNLIYYTNSIDRLKLLYDYMHDNISIKTYIDILQDINNKQYNDVKLYNGVYNTNVSEESHIYNISNNVDHIFQKFNFKLPIKVESGGVYKESS

SEQ ID NO: 8 - DNA, 4335 bp, Plasmodium falciparum
atgaaattgtttgattcattttttaaacatgctttgataagaataaataaaaggaatataatatatttgaatgccactaggtactattgtaataacataaattataatgctttgataaatttgttaaataagaaaaatgatataaataaagaaataaatgccttatattctttattagaaagactgtcaaattataagtacaaacaatataaagataagttgactctgaaaaataatataaacgatgaaattaaaataacaaatgctgataaaattaataatataaatattgaaagggatatgaatatttctcatttggatcatcatcataataatcatcatcataataataatcatcataatattaatcataataatcatcatcataataatcatcataatattaatcatcataataatcatcataataatcatcataataataatcattttaatgattataaaaaactaattgataattggaaaaatgataaaataaaaatatttataagctggtgtcccgaaattgttgaagataaatataagtccaaatgtttctctataccaacttatatcacatttcatattgttataactaataatgatattaagttgaataaccttttgcataattctcatgaatatgatgattggaactttaataaaataatacaaacaataaataatcaaaacaatttaaaagataaagaaaaggaaaaagaaaatggacaacaacattcacaagaatatattggaaattgtaaaaagggagaatctgaaataccatcatatgatttcaaagaatctttattagaacacataaatgaatcttcacaattaaatcattccatattatcgcacaaaacaaaagaacaaacccatcatactaataataatataaatggtaattataataatgatgaacatattgaagaagagggaaaggcaaaaacaaaacaaaacaaaacaaaaaatagcataatagaggaaaaaaaaaaaaagacaaaaaaaaaaaaagatgaagaatctcataatgatataataaattatactataaagaaaaaaacaaatacaaataattcattatataatatagaatcaatcttaaatataccaaaaacttatgaacctaatatacattatgataaatgtatacataaagaacaaaatcatatttttttcttttcctttaatatatccgaattaataattaatgatcaagtaaaaacaaaattgaatgaatgtattcaacaaaattttataaaacaaaatatatcaaatatacatatggatgatctttttttatatgttgtatatgattataaaaatttaatacatatatttaataatattaatttaaaattaataaatattaataatatatttgatatatatattattagttcactaatacaattagttcaaagaggggaaaaattacaaaatgtgtataatgaatatttaaatgttaaacataaaattcttatacctaataaaattaatgatatacaaaatttaagtcttcataatttttcgtatttttcaaagtttgctcctgaatttagcgatgtcatatcagcaaagtttgggttatatggttggggaaaatatcaaaagaaaaaagataagaaaaataaaaaacaaactgaaaatcatgaaaataatgaaaattatgaaaataatgaatacggtaaaaataatgaatatggtaaaaataatgaatatggtaaaaataatgaatatggtaaaaataatgaatatggtaaaaataatgaatatggtaaaaataatgaatatggtaaaaataatgaatatggtaaaaataatgtacacaatgacgatacatatatggacatatcaaatgagagaaagaataagaagagtaaagaagtcaaaaacaaaaaaaagatggagaaaaaaaacaaagtagaaaaagaaaaacaaaattatttaagttttactcctcataatataaataatcttcaagatattaaaaaacttgtatttgggaataaaagaaatatatcagatattacagaagaagataatatatgttatagtatatcacgaaattgttgtttaattttactatttgaatatttcataaataaattagaacataatatcaacatactaaatttatacatcaaagttgaacaaccattaatattatgtataagtcatatagaggaaaaaggaatcttcttgaatcaaaataaaattgaagaaatacaaaaaaaatcagatgaccctttaatatataaaaatgaaattgaagaattatgtaaatgtaatattaatttgaattcatccaagcaagtctcttcattgatatataaacaattattagacatatccattagcacagatcacacggaagaaaatatggaagatgcagatgaacatacagatcaccaggaagaagaacatgtaaatgatgataataatgaatgtgtagatcaattaaaagcatatactcaaacaaaggaaaaagaaagaaaagacatatataataataataataatgaaaataataaaaataataatgaaaattataattcaagtaaaaatcatcctttaataacaaatactaataacgatgatacatctacacttaatgcacaagatacatctgaccaacatgataattatataaatgaacataataattataataaatttataaaaaataaccctttttactataataataataataataataataataataataataataataatgataataataataatatttctaatcgtaatcttatgaacaatcttgtaaacattaattacacttcattatataataaaaagaaaaatagtcatccatatgatgaaaataataagttgtttttcctcaacagcagccataataattataataataataataataatattaacgaaatgagcagaaacaaaaatctacaaaccaataataagtcattaaaaattcttgtcgatgaaattgaaaaaagtaattatataaaagaaaaggaaaaagaaaaattaaaaaaaattattagaaatattaaattatatagagaatctaaaaaattagtacaaaattatatagaaaatctccctaaatatatacaaaaaaatacaaataaaatacattgtaattttaatcaaattggagcatcaacaggaagattatcttgtgatcaaccaaatttgcaaaatatacattcacgatttcgttgtgctatatcgttaaaaggtaaggaagaaaatgacacacatgataataataataataataataatataccacagattcatatatcaactaataatgtatcaaccaataatgtacccatgaatatcatgtcatctacatatcctttatataccatgaataaaaaaaatttaatcaccttcgattataaacaaatggaattatttgtcatggcatatctcagttttgatgaacaattattgaaattattaaattatagtgatgtatttatcgaaacagccaaagtattatttaatacaaatgatgttaccaatgaattaagaagaatgaccaaaactgttatatatggtatattatatggacaaactgaaaatggactagccaaaagtttattaattagcgatactttggctagtaacctaatagaaaacttttttcaattttttccaaacgtatatcgatttatgcaaatgcagaaatttttagtcaaacatatgaattgtgtttatacacttataggaaggaaaagaataatattaccaaacattaaaaataaatataggataagtatgaatacacctatacaaggatgtgcagcagatattatgaaattttctctcttgtcatgttttagtgttcttaataataatatatataataacaataaattattaaaaatgaataatataaatcctttaatcatacataaaaatcaagcctttttaaatccaactaatttaattttgcaagtacatgatgaattattattagaaagtgaacatgatgctaccaaatatataatacaactactaaatcctatattagaaaatgctttttataatttaatttattatacgaactctatagatagacttaaactattatatgattatatgcatgataatatttctatcaaaacatatatagatattttacaagatataaataacaaacaatataatgatgtaaaattatacaatggtgtatataatacaaatgtatcagaagaatcacacatatataatatatcaaataatgtggatcatatatttcaaaaatttaattttaagttgcctattaaagttgaatcaggcggagtctacaaggagtcttcataa

SEQ ID NO: 9 - DNA, 2142 bp, Plasmodium falciparum
aaaggtatttttctgaatcagaataaaattgaagaaattcagaagaaaagcgacgacccgctgatctataaaaacgaaattgaggaactgtgcaaatgcaacattaatctgaacagcagcaaacaggtttctagcctgatttataaacagctgctggatattagcatcagcaccgatcataccgaagaaaatatggaagatgccgatgaacataccgatcatcaagaagaggaacacgttaacgatgataataacgaatgcgtggatcagctgaaagcatatacccagaccaaagaaaaagagcgcaaagacatctacaataacaacaacaacgagaacaataaaaacaataacgaaaactataatagcagcaaaaatcatccgctgatcaccaataccaacaatgatgataccagcaccctgaatgcacaggataccagcgatcagcatgataactatatcaacgagcacaacaactacaataaatttatcaaaaacaacccgttctattataataacaacaacaataacaacaataacaataacaacaacaatgacaataacaacaatattagcaatcgcaatctgatgaataatctggtgaatatcaattataccagcctgtataataaaaagaaaaatagccatccgtatgatgaaaataacaaactgttttttctgaatagcagccataataactataacaataataacaataacattaatgaaatgagccgcaataaaaatctgcagaccaataataaaagcctgaaaatcctggtggatgagatcgaaaagagcaactatattaaagagaaagagaaagagaaactgaagaaaatcattcgcaatattaaactgtatcgcgaaagcaagaaactggtgcagaactatattgagaatctgccgaaatatattcagaagaacaccaataaaattcattgcaattttaatcagattggtgcaagcaccggtcgtctgagctgtgatcagccgaatctgcagaatattcatagccgttttcgttgtgccattagcctgaaaggtaaagaagaaaacgatacccacgacaataataataataataacaacattccgcagattcatattagcaccaataatgtgagcaccaataacgtgccgatgaatattatgagcagcacctatccgctgtacaccatgaataagaaaaacctgattacctttgattacaaacaaatggaactgtttgtgatggcctatctgagctttgatgaacagctgctgaaactgctgaattatagcgacgtgtttatcgaaaccgccaaagttctgtttaacaccaatgatgtgaccaatgaactgcgtcgtatgaccaaaaccgtgatttatggtattctgtatggccagaccgaaaatggtctggcaaaaagcctgctgattagcgataccctggcaagcaatctgatcgaaaacttttttcagttctttccgaacgtgtatcgctttatgcagatgcagaaatttctggtgaaacatatgaactgcgtgtacaccctgattggtcgtaaacgtattattctgccgaatattaaaaacaaatatcgcattagcatgaataccccgattcagggttgtgcagcagatattatgaaatttagcctgctgagctgctttagcgtgctgaataataacatctacaacaacaacaaactgctgaaaatgaataatatcaatccgctgattattcataaaaatcaggcctttctgaacccgaccaatctgattctgcaggttcatgatgagctgctgctggaaagcgaacatgatgcaaccaaatatattatccagctgctgaatccgattctggaaaatgccttttacaatctgatttattataccaacagcattgatcgcctgaaactgctgtatgattatatgcatgataatattagcattaaaacctatatcgatattctgcaggatatcaataacaaacagtataatgatgtgaaactgtataatggcgtgtacaataccaacgttagcgaagaaagccatatctataacatcagcaacaacgtggatcatatctttcagaaatttaattttaaactgccgattaaagtggaaagcggtggtgtttataaagaaagcagctaa

SEQ ID NO: 10 - DNA, 2142 bp, Plasmodium falciparum
aagggcatcttcctgaaccagaacaagatcgaggagatccaaaagaagtccgacgacccactcatctacaagaacgagatcgaggagctgtgcaagtgcaacatcaacctcaactccagcaagcaagtctcgtcactgatctacaagcagctcctggatatctccatcagcaccgaccacacggaggagaacatggaggacgcggatgagcacaccgaccatcaagaggaggagcatgtgaacgacgataacaacgagtgcgtcgatcaactcaaggcttacacccagacgaaggagaaggagaggaaggacatctacaacaacaacaacaacgagaacaacaagaacaacaacgagaactacaactcttccaagaaccacccactgatcaccaacacgaacaacgacgatacctccacgctcaacgcccaagacaccagcgatcagcacgacaactacatcaacgagcataacaactacaacaagttcatcaagaacaacccgttctattataacaacaacaacaacaacaacaacaacaacaacaacaacaacgataacaacaacaacatcagcaaccggaacctgatgaacaacctcgtgaacatcaactacacctcgctgtacaacaagaagaagaactcacacccatacgacgagaacaacaagctgttcttcctcaacagctcccacaacaactataacaacaacaacaacaacatcaacgagatgtctagaaacaagaacctccagaccaacaacaagtccctgaagatcctcgtcgacgagatcgagaagtccaactacatcaaggagaaggagaaggagaagctgaagaagatcatccggaacatcaagctgtacagagagtccaagaagctcgttcaaaactacatcgagaacctccccaagtacatccagaagaacaccaacaagatccactgcaacttcaaccaaatcggcgcttctacggggagactgtcctgcgaccaacctaacctccagaacatccattcacggttcagatgcgcgatctctctcaagggcaaggaggagaacgatacccacgacaacaacaacaacaacaacaacataccgcagatccatatcagcaccaacaacgtgtcgacgaacaacgtcccgatgaacatcatgtcatctacctacccgctgtacacgatgaacaagaagaacctcatcaccttcgactacaagcagatggagctgttcgtcatggcttacctctcgttcgatgagcagctcctgaagctcctgaactacagcgacgtgttcatcgagaccgccaaggtcctgttcaacaccaacgacgttacgaacgagctcaggcgcatgaccaagaccgtgatctacggcatcctgtacgggcaaaccgagaacgggctcgctaagtcgctcctgatctcagacacgctggcctccaacctcatcgagaacttcttccagttcttccccaacgtctacaggttcatgcaaatgcagaagttcctcgtcaagcacatgaactgcgtttacaccctgatcggcaggaagcgcatcatcctcccaaacatcaagaacaagtaccgcatcagcatgaacaccccgatccaagggtgcgccgcggacatcatgaagttctcgctcctgtcatgcttctctgttctgaacaacaacatctacaacaacaacaagctcctgaagatgaacaacatcaaccccctgatcatccacaagaaccaagcgttcctcaaccctaccaacctgatcctccaggtgcacgatgagctcctgctcgagtccgagcatgacgcgacgaagtacatcatccagctgctcaaccctatcctggagaacgctttctacaacctcatctactacaccaactccatcgaccggctcaagctgctctacgattacatgcacgacaacatctcaatcaagacgtacatcgatatcctgcaagacatcaacaacaagcagtacaacgatgttaagctctacaacggcgtttacaacaccaacgtgtccgaggagagccacatctacaacatctccaacaacgtggaccatatcttccaaaagttcaacttcaagctgccaatcaaggtggagtcggggggcgtctacaaggagtcgtcgtga

(19) United States
(12) Patent Application Publication
(10) Pub. No.: US 2013/0102499 A1
(43) Pub. Date: Apr. 25, 2013

(54) METHOD FOR DETERMINATION OF ACTIVITY OF MITOCHONDRIAL DNA POLYMERASE OF FALCIPARUM MALARIA, AND METHOD FOR SCREENING FOR ANTI-MALARIA COMPOUND

(71) Applicants: Narie Sasaki, Nagoya-shi (JP); Kimiko Murofushi, Tokyo (JP); Katsura Maeda, Tokyo (JP)

(72) Inventors: Narie Sasaki, Nagoya-shi (JP); Kimiko Murofushi, Tokyo (JP); Katsura Maeda, Tokyo (JP)

(73) Assignees: OCHANOMIZU UNIVERSITY, Tokyo (JP); NATIONAL UNIVERSITY CORPORATION NAGOYA UNIVERSITY, Nagoya-shi (JP)

(21) Appl. No.: 13/806,881

(22) PCT Filed: Jun. 17, 2011
(86) PCT No.: PCT/JP2011/063965

§ 371 Date: Dec. 26, 2012

(30) Foreign Application Priority Data
Jun. 28, 2010 (JP) 2010-145907

(51) Int. Cl. C12Q 1/68

(52) U.S. Cl. CPC C12Q 1/68
USPC 506/11; 435/6.1

(57) ABSTRACT

An object is to provide a means which is useful for the development of an anti-malaria agent. It was found that a mitochondrial DNA polymerase of falciparum malaria shows a bivalent iron ion requirement. Thus, disclosed is a method for measuring the activity of a DNA polymerase, including the steps of: (1) incubating a solution containing a bivalent iron ion, a mitochondrial DNA polymerase of falciparum malaria, template DNA, and at least one deoxyribonucleoside triphosphate or deoxyribonucleoside triphosphate derivative; (2) detecting the synthesized double-stranded DNA; and (3) calculating the activity of the DNA polymerase from the result of the detection carried out in step (2).

### TECHNICAL FIELD

The present invention relates to a test method (assay) using a mitochondrial DNA polymerase of falciparum malaria. Specifically, the invention relates to a method for measuring the activity of the mitochondrial DNA polymerase, and a method for screening for an anti-malaria compound, using an inhibition activity to the mitochondrial DNA polymerase as an indication. The present application claims priority based on Japanese Patent Application No. 2010-145907 filed on Jun. 28, 2010, and the entire content of the patent application is incorporated herewith by its reference.

### BACKGROUND ART

Malaria is widespread in tropical regions and one of serious infections. The damage is enormous and it is said that 3 to 5 hundred million people are infected in a year mainly in Africa and 1 to 2 million people are died for the infection (according to an estimate by WHO, 2005). Malaria is recognized as one of the three major infections in the world in concurrence with AIDS and tuberculosis, and becomes a serious problem particularly in progress in developing countries. Also in Japan, cases of infections by travel to a malaria pervasive region and development after return home are increasing and 100 or more cases of the infection have reported every year, and several fatal cases are included in a year (Quarantin Information Office, Ministry of Health, Labour, and Welfare Japan).

The pathogenic organism is malaria parasite (Plasmodium spp.) that is a single-cell organism and transmitted by an anopheles mosquito (Anopheles spp.). The malaria parasite belongs to the phylum Apicomplexa, the class Sporozoea, the subclass Coccidia, and classified into the line called Alveolata from the fine structure and a molecular phylogenetic analysis. In other microorganisms that belong to the line, dinoflagellates are known, and recently, an organelle called apicoplast that is a trace of a plastid having an original DNA has been found also from a malaria parasite (Non-patent Document 1). Also from the fact, ancestors of apicomplexa all of which are parasites are considered to be phototrophic organisms same as dinoflagellates. Human pathogens are classified into four types, which are falciparum malaria (Plasmodium falciparum), tertian fever malaria parasite (P. vivax), quartan fever malaria parasite (P. malariae), and ovale malaria parasite (P. ovale), and the symptom of malaria caused by falciparum malaria is particularly severe.

The life cycle of falciparum malaria (hereinafter abbreviated as “malaria parasite”) was shown in FIG. 3. When a female anopheles mosquito infected with a malaria parasite sucks human blood, the malaria parasite (sporozoite) enters into the human blood vessel with mosquito's saliva. The infectious parasite entered into the blood vessel transfers to a hepatic cell and proliferates in the hepatic cell for 7 to 10 days. When the parasite sufficiently proliferates and matures, the parasite breaks the hepatic cell and is released into the blood. The released parasite enters into an erythrocyte, and grows to a ring (ring form), a trophozoite and a schizont, and over twenty merozoites are newly born. During the growing, the parasite decomposes hemoglobin in the erythrocyte and takes the obtained amino acid as nourishment. Then, the erythrocyte is destructed and the merozoites are released and, during the releasing, fever specific to malaria is caused. Parasites released in the blood enter into as many erythrocytes as possible and repeat proliferation. A part of the merozoites become gametocytes and are taken in the mosquito's stomach when the anopheles mosquito sucks blood from this patient. A parasite taken in the body of the mosquito is sexually divided to form a sporozoite, and transfers to the mosquito's salivary gland. The mosquito sucks another human blood and infection is thus spreading.

Currently, there is no vaccine against malaria parasites and the infection cannot be prevented with a drug. Several anti-malaria agents have been developed and quinine has been used as a typical treating agent for a long time but has a problem such as very strong side effects. Drugs such as chloroquine, mefloquine, fansidar, and primaquine are developed and, in particular, chloroquine is used as a preventive drug or a drug that is tried in an initial stage of the treatment in many cases due to fewer side effects than the other drugs. However, parasites having resistance to malaria treating agents including chloroquine have started to spread in recent years, which becomes a serious problem. Other than such parasites, anopheles mosquitoes having resistance to pesticides appear and development of a novel drug is thus urgently requested. Targets for the drug development, which have drawn attention, are malaria parasite organelles such as apicoplasts and mitochondria.

Both of mitochondria and apicoplasts each have original DNA, and constituted with both of a gene product coded therein and a gene product coded in a nucleus (FIG. 4). The apicoplast is an organelle that is considered to be a trace of a plastid as described above, and cannot perform photosynthesis. About 500 genes among nuclear genomes of a malaria parasite code for a protein targeting an apicoplast. It has been known that a DNA gyrase (topoisomerase II) is required for replication of apicoplast DNA (hereinafter abbreviated as “apDNA”) (Non-patent Documents 2 and 3). In addition, a DNA polymerase PF14_{—}0112 (POMI/Pfprex), which performs replication of apDNA, was recently identified (Non-patent Document 4). However, the specific replication mechanism is not clarified.

On the other hand, a DNA polymerase relating to replication of mitochondrial DNA (hereinafter abbreviated as “mtDNA”) of a malaria parasite has not been identified yet. A particular replication form called the rolling circle type is considered to be used and the details of the replication mechanism and proteins relating to replication and transcription are not identified at all. A DNA polymerase γ-like enzyme that is an enzyme relating to replication of mtDNA has been partially purified from a malaria parasite so far (Non-patent Document 5). It has been revealed that the partially purified enzyme has properties similar to a known mammalian DNA polymerase γ (pol γ) such as (1) aphidicolin resistance and (2) N-ethylmaleimide (NEM) sensitivity; on the contrary, the enzyme has different properties such as (3) ddTTP resistance and (4) PMEApp resistance (Non-patent Document 5). However, isolation and purification of the mtDNA polymerase from malaria parasites are not succeeded due to difficulties in mass culture of malaria parasites and purification of mitochondria.

### PRIOR ART DOCUMENTS

### Non-Patent Documents

Non-patent Document 1: Wilson et al. Infect. Agents Dis. 3: 29-37, 1994
Non-patent Document 2: Fichera et al. Nature 390: 407-409. 1997
Non-patent Document 3: Weissig et al. DNA Cell Biol. 16: 1483-1492, 1997
Non-patent Document 4: Seow et al. Molecular & Biochemical Parasitology 141: 145-153, 2005
Non-patent Document 5: Charavalitshewinkoon-Permitr et al. Parasitology International 49 279-288, 2000

### SUMMARY OF INVENTION

### Problems to be Solved by the Invention

An object of the present invention is to provide a means (tool) which is useful for development, and the like, of an anti-malaria agent.

### Means for Solving the Problems

The present inventors promoted studies focusing on a mtDNA polymerase of a malaria parasite in order to solve the above descried problems. A mitochondrion of a malaria parasite is an essential organelle for survival of the malaria parasite and is also considered to be effective as a target of novel drug discovery due to the uniqueness of the structure, and it is thus very important to clarify the replication mechanism of mtDNA also from the viewpoint of medicine.

In recent years, a DNA polymerase analogous to the E. coli DNA polymerase I (pol I) was identified from a higher plant (Christensen et al. Plant Cell. 17(10): 2805-2816 2005, Kimura et al. Nucleic Acids Res. 1; 30(7): 1585-1592 2002, Mori et al. Biochem Biophys Res Commun. 19; 334(1): 43-50. 2005, Ono et al. Plant Cell Physiol. 48(12): 1679-1692. 2007). A mitochondrial DNA polymerase (PpPolA) analogous to the DNA polymerase I was identified from Physarum polycephalum also in the research group of the present inventors.

The PpPolA is the most analogius to the DNA polymerase I and was expected to be a primitive mitochondrial DNA polymerase. Thus, homology search, local existance analysis, and the like were carried out based on the sequence of PpPolA to aim for finding of a mitochondrial DNA polymerase of a malaria parasite. As a result, identification of a sequence having high possibility to function as the mitochondrial DNA polymerase of a malaria parasite was succeeded. Then, after trial and error, expression of the sequence was also succeeded by use of a cell-free synthesis system. As a result of examining characteristics of the expressed protein, it was surprisingly revealed that the protein requires a bivalent iron ion (Fe²⁺) to exert its activity. That is, it was found that the protein has an unexpected characteristic such as showing the requirement of a bivalent iron ion, not bivalent metallic ions such as magnesium and manganese, which is different from other DNA polymerases. This characteristic was also confirmed by an experiment using a mitochondrial fraction.

As described above, essential conditions for exhibition of the malaria parasite activity of a mitochondrial DNA polymerase were found from studies made by the present inventors. The achievement enabled measuring the activity of the enzyme in vitro, that is, “establishment of the activity measurement system of the enzyme”. The activity measurement system can be used as a research tool for granted and also as a means for screening for an anti-malaria compound. That is, the activity measurement system is also conducive to a technique used for development of an anti-malaria agent and its value is infinite. In addition, as a result of specific studies on characteristics of the enzyme, beneficial findings regarding optimizing the activity measurement system such as concentration dependency and pH dependency, which relate to Fe²⁺, were also obtained.

The present inventions listed below are mainly based on the above described achievement.

[1] A method for measuring the activity of a DNA polymerase, including the following steps of (1) to (3):

(1) incubating a solution containing a bivalent iron ion, a mitochondrial DNA polymerase of falciparum malaria, template DNA, and at least one deoxyribonucleoside triphosphate or deoxyribonucleoside triphosphate derivative;

(2) detecting the synthesized double-stranded DNA; and

(3) calculating the activity of the DNA polymerase from the result of the detection carried out in step (2).

[2] The method for measuring the activity of a DNA polymerase according to [1], wherein the mitochondrial DNA polymerase includes any one of sequences set forth in SEQ ID NOs. 1 to 7 or partially altered sequences thereof and shows the DNA polymerase activity.

[3] The method for measuring the activity of a DNA polymerase according to [1] or [2], wherein the mitochondrial DNA polymerase is a protein prepared in a cell-free synthesis system.

[4] The method for measuring the activity of a DNA polymerase according to any one of [1] to [3], wherein the template DNA is activated double-stranded DNA, or a combination of one-stranded DNA or a polynucleotide chain constituted with one kind of deoxyribonucleotide, and a complementary primer thereof.

[5] The method for measuring the activity of a DNA polymerase according to any one of [1] to [4], wherein the detection of the double-stranded DNA is carried out by fluorescence staining specific to double-stranded DNA.

[6] The method for measuring the activity of a DNA polymerase according to any one of [1] to [5], wherein the concentration of the bivalent iron ion in the solution is 5 mM to 15 mM.

[7] The method for measuring the activity of a DNA polymerase according to any one of [1] to [6], wherein the pH of the solution is 7 to 8.

[8] The method for measuring the activity of a DNA polymerase according to any one of [1] to [7], wherein the mitochondrial DNA polymerase is thermally pretreated in the temperature condition from 50° C. to 90° C.

[9] The method for measuring the activity of a DNA polymerase according to any one of [1] to [8], wherein the incubation in the step (1) is carried out in the presence of a test substance.

[10] A method for screening for an anti-malaria compound including the following steps of (i) to (iii):

(i) incubating a solution containing a bivalent iron ion, a mitochondrial DNA polymerase of falciparum malaria, template DNA, and at least one deoxyribonucleoside triphosphate or deoxyribonucleoside triphosphate derivative in the presence of a test substance;

(ii) detecting the synthesized double-stranded DNA; and,

(iii) determining effectiveness of the test substance based on the result of the detection carried out in step (ii), wherein inhibition of double-stranded DNA synthesis is indicative of effectiveness.

[11] The screening method according to [10], wherein a sample (control group) incubated under the same conditions as in the step (i) except for the absence of a test substance is prepared and effectiveness in the step (iii) is determined by comparing to the detection result for the control group in the step (ii).

[12] The screening method according to [10] or [11], further including a step of evaluating an inhibition activity to a nuclear DNA polymerase of falciparum malaria for a test substance which showed effectiveness in the step (iii).

[13] The screening method according to any one of [10] to [12], further including a step of confirming that a test substance which showed effectiveness in the step (iii) shows no inhibition activity to a human DNA polymerase.

### BRIEF DESCRIPTION OF DRAWINGS

FIG. 1 shows one example of the method for measuring the activity of a mitochondrial DNA polymerase of a malaria parasite. Double-stranded DNA is detected using a radioactive isotope. In addition, quenching is prevented by adding gallic acid, or the like, to form trivalent iron into a complex.

FIG. 2 shows one example of the method for measuring the activity of a mitochondrial DNA polymerase of the malaria parasite. Double-stranded DNA is detected using fluorescence.

FIG. 3 is a view showing a life cycle of a malaria parasite. The encirclement in the lower column shows giemsa stained images of a malaria parasite in each stage.

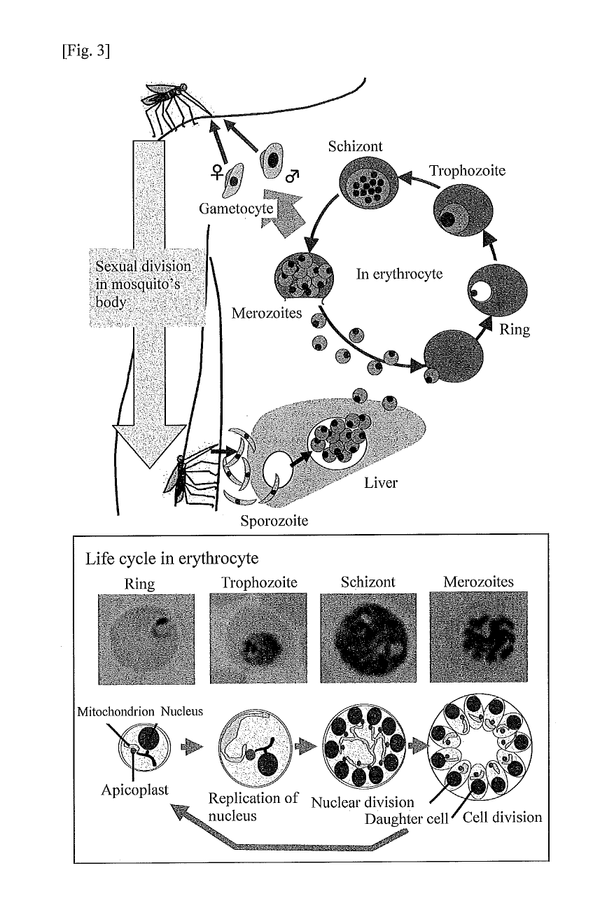

FIG. 4 is a view showing structures of organelles of a malaria parasite and organelle DNA.

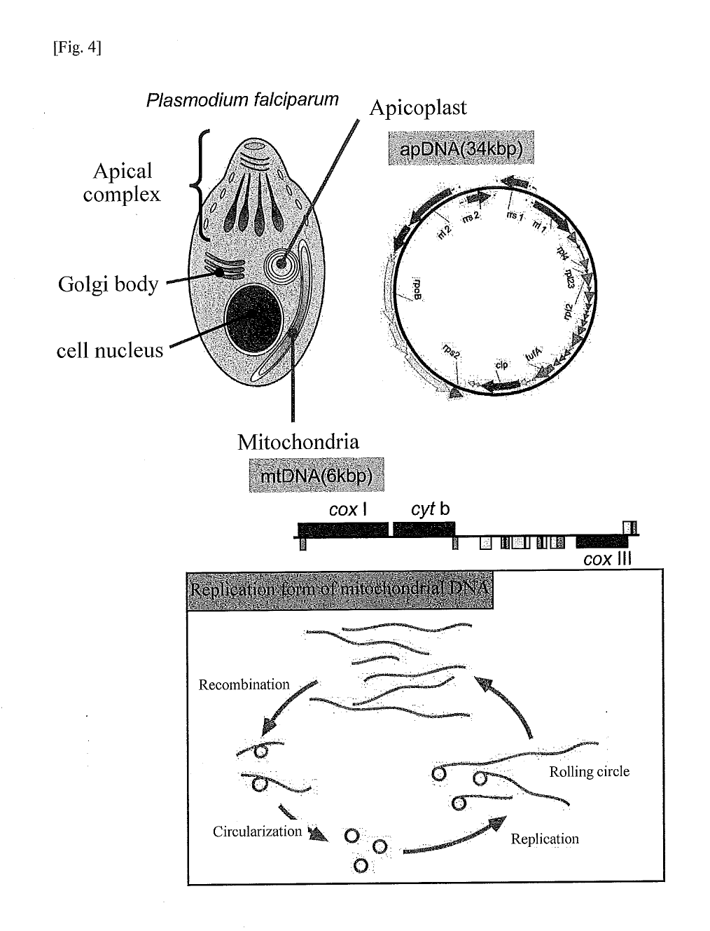

FIG. 5 is a view showing the full length amino acid sequence (SEQ ID No. 1) of PFF1225c.

FIG. 6 is a schematic view showing a wheat germ cell-free type protein expression system.

FIG. 7 shows results of an experiment of protein expression using the wheat germ cell-free protein expression system. a shows a region where expression was tried, and b shows results of western blotting using an anti-His-tag antibody of a protein after expression. * shows a band of a protein in each region to be estimated.

FIG. 8 shows results of a study of an ion requirement using PFF1225c (C1 fragment).

FIG. 9 shows results of an analysis of the polymerase activity of PFF1225c (C1 fragment). a is a graph showing an optimum iron ion concentration. b is a graph showing an optimum pH of an enzyme. c is a graph showing thermal stability of an enzyme. High activities were shown when the Fe²⁺ concentration was from 5 mM to 15 mM (the optimum concentration is 10 mM). Preferable activities were also shown when a pH was from 7 to 8 (the optimum pH is 7.5). On the other hand, when a thermal treatment was carried out at a temperature from 50° C. to 90° C., the activity increased, and the highest activity was shown in a thermal treatment at 70° C.

FIG. 10 shows results of a study of an ion requirement using a human mitochondrial DNA polymerase γ.

FIG. 11 shows results of a study of sensitivity to an inhibitor using PF1225c (C1 fragment). a is a table showing sensitivity of each DNA polymerase to an inhibitor. b is a graph showing sensitivity to aphidicolin (Aphidicolin). c is a graph showing sensitivity to NEM. d is a graph showing sensitivity to ddTTP.

FIG. 12 shows results of a study of sensitivity of various DNA polymerases to chloroquine. a is a graph showing sensitivity of PF1225c (C1 fragment). b is a graph showing sensitivity of a Physarum polycephalum mitochondrial DNA polymerase (PpPoIA). c is a graph showing sensitivity of a human mitochondrial DNA polymerase γ.

FIG. 13 shows results of a study of sensitivity of various DNA polymerases to suramin. a is a graph showing sensitivity of PF1225c (C1 fragment). b is a graph showing sensitivity of a Physarum polycephalum mitochondrial DNA polymerase (PpPolA). c is a graph showing sensitivity of a human mitochondrial DNA polymerase γ.

FIG. 14 shows results of the measurement of the DNA polymerase activity using a malaria parasite mitochondrial fraction.

### DESCRIPTION OF EMBODIMENTS

### Terms

A mitochondrial DNA polymerase is abbreviated as a “mtDNA polymerase” in the specification as described above. Note that when a “mtDNA polymerase” is described herein without particular notification, it means a mtDNA polymerase of a malaria parasite.

### 1. Method for Measuring the Activity of DNA Polymerase

The first aspect of the present invention relates to a method for measuring the activity of a mitochondrial DNA polymerase (mtDNA polymerase) of a malaria parasite (Plasmodium spp.). The activity measurement method of the present invention is useful as a research tool for a mtDNA polymerase of a malaria parasite. In addition, the method is also useful as a means for searching a substance showing an inhibition activity to the mtDNA polymerase. Such a substance showing an inhibition activity to the mtDNA polymerase is expected to be used and applied as an anti-malaria agent or a leading compound of the anti-malaria agent.

The following steps (1) to (3) are carried out in the activity measurement method of the present invention:

(1) incubating a solution containing a bivalent iron ion, a mitochondrial DNA polymerase of falciparum malaria, template DNA, and at least one deoxyribonucleoside triphosphate or deoxyribonucleoside triphosphate derivative;
(2) detecting the synthesized double-stranded DNA; and
(3) calculating the activity of the DNA polymerase from the result of the detection carried out in step (2).

It was revealed from studies made by the present inventors that a mtDNA polymerase of a malaria parasite shows a bivalent iron ion requirement. Based on the finding, the reaction is carried out in the step (1) in the condition of the presence of a bivalent iron ion in the solution. This point is the most significant characteristic of the present invention. For example, by adding and dissolving a compound that generates bivalent iron ions, such as iron chloride (FeCl₂) and iron sulfate (FeSO₄), a reaction solution satisfying the condition can be prepared. The amount of the bivalent iron ion in the reaction solution, that is, the concentration of the bivalent iron ion in the reaction solution is not particularly limited as long as the activity of the mtDNA polymerase is detected. However, according to the finding obtained by studies made by the present inventors (see examples described below), the concentration of the bivalent iron ion is preferably set within the range from 5 mM to 15 mM. The concentration of the bivalent iron ion is more preferably set to about 10 mM. Note that, in order to attempt reduction of an error of the measurement and improvement in reproducibility, oxidation of the bivalent iron ion in the reaction solution is desirably prevented by, for example, using degassed water.

A mtDNA polymerase that is the primary material of the enzyme reaction, template DNA that provides an initiation point of DNA synthesis, and a substrate (material) for DNA synthesis are included in the reaction solution in addition to a bivalent iron ion. These elements will be described respectively below.

(a) mtDNA Polymerase

A mtDNA polymerase may not have the full length as long as the DNA polymerase activity is shown. In other words, the mtDNA polymerase may be a partial sequence as long as a region that is necessary for the DNA polymerase activity is included. One example of a sequence of a mtDNA polymerase (PFF1225c) is set forth in SEQ ID No. 1 in the sequence listing. The sequence is an annotated sequence as a DNA polymerase I-like protein in the public database (NCBI, Protein Database, DEFINITION: DNA polymerase 1, putative [Plasmodium falciparum 3D7]., ACCESSION: XP 966236). A nucleotide sequence coding for the amino acid sequence (coding region of a gene) is set forth in SEQ ID No. 8. In addition, examples of partial sequences containing a DNA polymerase domain (pol Ac) of the amino acid sequence are set forth in SEQ ID Nos. 2 to 7. Regions corresponding to these partial sequences are shown as follows.

SEQ ID No. 2: No. 104 amino acid to No. 1444 amino acid in the sequence of SEQ ID No. 1

SEQ ID No. 3: No. 276 amino acid to No. 1444 amino acid in the sequence of SEQ ID No. 1

SEQ ID No. 4: No. 426 amino acid to No. 1444 amino acid in the sequence of SEQ ID No. 1

SEQ ID No. 5: No. 618 amino acid to No. 1444 amino acid in the sequence of SEQ ID No. 1

SEQ ID No. 6: No. 732 amino acid to No. 1444 amino acid in the sequence of SEQ ID No. 1 SEQ ID No. 7: No. 990 amino acid to No. 1444 amino acid in the sequence of SEQ ID No. 1

An applicable mtDNA polymerase is not particularly limited to the above described examples (SEQ ID Nos. 1 to 7) as long as the DNA polymerase activity is shown. For example, a mtDNA polymerase made of a sequence obtained by partially altering any of the sequences of the above described examples and showing the DNA polymerase activity (typically, pol Ac is contained) can be used as the mtDNA polymerase in the same manner. “Partially altering” herein refers to occurrence of change in an amino acid sequence by deletion or replacement of 1 to several amino acids constituting the amino acid sequence, or addition or insertion of 1 to several amino acids, or a combination thereof. A position of mutation in an amino acid sequence is not particularly limited, and mutation may occur in a plurality of positions. A plurality of herein means the number corresponding to, for example, 10% or less of the whole amino acids constituting the amino acid sequence, preferably the number corresponding to 5% or less of the whole amino acids, and more preferably the number corresponding to 1% or less of the whole amino acids. Such alteration is preferably performed in a region other than pol Ac. Note that in the case where a region other than pol Ac is a region to be altered, significant alteration can be accepted since effects on the DNA polymerase activity is less (or substantially no effect).

A mtDNA polymerase can be prepared, for example, by using a known protein synthesis system. However, when a general E. coli expression system is used, in the light of difficulty in expression of the protein by E. coli (see examples described below), the protein may be expressed after adjustment and modification of a sequence in consideration of frequency in use of a codon. As a specific example of a DNA sequence that can be used for synthesis of a mtDNA polymerase (that is, DNA coding for the mtDNA polymerase), a DNA sequence optimized for expression in E. coli (corresponding to the amino acid sequence of SEQ ID No. 6) is set forth in SEQ ID No. 9.

A mtDNA polymerase is preferably prepared by using a cell-free synthesis system. In the present invention, the cell-free synthesis system (cell-free transcription system, cell-free transcription/translation system) refers to synthesizing mRNA or a protein in vitro from nucleic acid (DNA or mRNA) coding for the mRNA or the protein, which is a template, by using a ribosome or a transcription/translation factor derived from a living cell (or obtained in a genetic engineering technique) without using a living cell. In the cell-free synthesis system, a cell extraction obtained by purifying a cell homogenate according to necessity is generally used. The cell extraction generally contains a ribosome, various factors such as an initiation factor and various enzymes such as tRNA, which are necessary for protein synthesis. When synthesis of a protein is carried out, other substances necessary for the synthesis of a protein such as energy sources including various amino acids, ATP and GTP, and phosphocreatine are added to this cell extraction. Needless to say, ribosome and various factors and/or various enzymes, and the like, which are separately prepared, may be complemented in the protein synthesis according to necessity.

Development of a transcription/translation system in which each molecule (factor) necessary for the protein synthesis is reconstructed has also been reported (Shimizu, Y. et al.: Nature Biotech., 19, 751-755, 2001). In this synthesis system, a gene made of 31 types of factors including three initiation factors constituting a bacterial protein synthesis system, three extension factors, four factors relating to termination, 20 aminoacyl tRNA synthesis enzymes connecting each amino acid to tRNA, and methionyl tRNA formyl transferase is amplified from an E. coli genome, and a protein synthesis system is reconstructed in vitro using these genes. Such a reconstructed synthesis system may also be used in the present invention.

The term “cell-free protein synthesis system” is exchangeably used with a cell-free transcription/translation system, an in vitro translation system, or an in vitro transcription/translation system. In the in vitro translation system, RNA is used as a template and a protein is synthesized. For template RNA, total RNA, mRNA, in vitro transcription products, and the like are used. In the other in vitro transcription/translation system, DNA is used as a template. The template DNA should contain a ribosome bonding region, and preferably includes a suitable terminator sequence. Note that, in the in vitro transcription/translation system, a condition of adding a factor necessary for each reaction is set so as to continuously progress a transcription reaction and a translation reaction.

The cell-free protein synthesis system has the following advantages. Firstly, operation property is preferable since there is no need to keep a living cell and the system has high flexibility. Therefore, a synthesis system in which various modifications and decorations are performed according to properties of a desired protein can be designed. Then, a toxic protein in a cell to be used basically cannot be synthesized in a cell-based synthesis, but such a toxic protein can also be produced in the cell-free system. Furthermore, since many kinds of proteins are simultaneously and quickly synthesized, high throughput synthesis is facilitated. An advantage such as easy separation and purification of a produced protein is also provided, which advantageously works for high throughput synthesis. In addition, the cell-free protein synthesis system has an advantage such that an unnatural protein can also be synthesized by incorporating an unnatural amino acid.

The following cell-free protein synthesis systems are currently widely utilized. That is, the cell-free protein synthesis systems include the E. coli S30 extraction system (procaryotic cell system), the wheat germ extraction system (eucaryotic cell system), and the rabbit reticulocyte lysate system (eucaryotic cell system). These systems are commercially available as kits, and can be easily used.

Historically, development of the E. coli S30 extraction system is the oldest, and various proteins have been tried synthesizing by use of this system. An E. coli 30S fraction is prepared through steps of collection of E. coli and fracture of the bacterial bodies, and purification. Preparation of an E. coli 30S fraction and a cell-free transcription/translation reaction can be carried out in reference to the method by Pratt et al. (Pratt, J. M.: Chapter 7, in “Transcription and Translation: A practical approach”, ed. by B. D. Hames & S. J. Higgins, pp. 179-209, IRL Press, New York (1984)) and the method by Ellman et al. (Ellman, J. et al.: Methods Enzymol., 202, 301-336 (1991)).

The wheat germ extraction system has an advantage such that a high quality eukaryotic protein can be effectively synthesized and is thus frequently used when an eukaryotic protein that is hardly synthesized in the E. coli S30 extraction system is synthesized. It was recently reported that a synthesis system with high efficiency and stability is established by preparing an extraction from an embryo obtained by washing and removing an endosperm component of a seed, which has drawn attention (Madin, K. et al.: Proc. Natl. Acad. Sci. USA, 97: 559-564, 2000). Then, technical developments such as a mRNA untranslation sequence having a high translation promoting ability, a protein synthesis method for a multi-item functional analysis using PCR, and establishment of a special high expression vectors are carried out (Sawasaki, T. et al.: Proc. Natl. Acad. Sci. USA, 99: 14652-14657, 2002), and applications to various fields are expected.

A wheat germ extraction can be obtained by grinding a wheat germ to be centrifuged, thereafter separating the supernatant liquid with gel filtration. The translation reaction can refer to the method by Anderson et al. (Anderson, C. W. et al.: Methods Enzymol., 101, 638-644 (1983)). A modified method has also been reported and can refer to, for example, the method by Kawarasaki et al. (Kawarasaki, Y. et al.: Biotechnol. Prog., 16, 517-521 (2000)) and the method by Madin et al. (Madin, K. et al.: Proc. Natl. Acad. Sci. USA, 97: 559-564, 2000). Other than the above, the wheat germ extraction system can refer to WO 00/68412 A1, WO 01/27260 A1, WO 2002/024939 A1, WO 2005/063979 A1, JP-A No. 6-7134, JP-A No. 2002-529531, JP-A No. 2005-355513, JP-A No. 2006-042601, JP-A No. 2007-097438, JP-A No. 2008-029203, and so on.

The rabbit reticulocyte lysate system is suitable for globulin production. A rabbit reticulocyte lysate is obtained through intravenously injecting phenylhydrazine to a rabbit for several days to make the rabbit in an anemic condition and taking blood after a predetermined period (for example, 8 days later), thereafter an ultracentrifugation treatment from the hemolyzed solution. A method for preparing the rabbit reticulocyte lysate can be carried out by reference to the method by Jackson and Hunt (Jackson, R. J. and Hunt, T.: Methods Enzymol., 96, 50-74 (1983)).

A cell-free synthesis system that can be used in carrying out the present invention is not limited to the above descried system, for example, systems constructed based on extractions of bacteria other than E. coli and plants other than wheat, extraction derived from insects, extractions derived from animal cells, or genomic information may be utilized.

For a specific example of a DNA sequence capable of being used in synthesis of a mtDNA polymerase by using a cell-free synthesis system (that is, DNA coding for mtDNA polymerase), a DNA sequence optimized for the wheat germ extraction system (corresponding to the amino acid sequence of SEQ ID No. 6) is set forth in SEQ ID No. 10. Increase in about 1.5 to 2 times of an expression amount was observed due to optimization.

By the way, as shown in examples described later, as a result of the study on pH dependency of a mtDNA polymerase, the peak of the activity was shown from pH 7 to 8 (optimum pH was 7.5). Thus, a pH of a reaction solution is preferably set from 7 to 8, and more preferably set to about 7.5.

As shown in examples described below, a surprising phenomenon such as improving the activity of the mtDNA polymerase by thermally treating the mtDNA polymerase was observed. Based on this finding, in one embodiment of the present invention, a mtDNA polymerase that is thermally pretreated in the temperature condition from 50° C. to 90° C. is used. The temperature condition of the thermal treatment is more preferably from 60° C. to 80° C., and the most preferably at about 70° C. The time for the thermal treatment is, for example, from 1 minute to 1 hour, preferably from 2 minutes to 30 minutes, and more preferably from 3 minutes to 15 minutes. Use of a mtDNA polymerase having an enhanced activity as described above results in effects such as improvement of measurement sensitivity, shortening of a measurement time, and decrease in a use amount of an enzyme. Note that desired effects cannot be sufficiently exerted when the temperature of the thermal treatment is too low, or the treatment time is too short. On the contrary, when the temperature of the thermal treatment is too high, or the treatment time is too long, deactivation of an enzyme can be caused.

### (b) Template DNA

Template DNA provides an initiation point of DNA synthesis. Template DNA capable of realizing high precision and credibility with maintaining simplicity may be used. Activated double-stranded DNA can be exemplified as template DNA satisfying the condition. The activated double-stranded DNA is obtained by treating suitable DNA (such as salmon sperm DNA and bovine thymus DNA) and providing a nick (cut). A deoxyribonuclease I treatment, a thermal treatment, sonication, and the like are used for activation. Another example of preferable template DNA is obtained by annealing a primer with a suitable length to one-stranded DNA. A specific example includes a combination of a polynucleoside chain (e.g., polyadenylic acid) constituted with one kind of deoxyribonucleoside and a primer complementary to the polynucleoside chain (e.g., oligo(dT) primer). Materials providing an initiation point of synthesis of a double-stranded DNA chain are comprehensively expressed as “template DNA” in the specification as typically represented by the above described two examples.

### (c) Substrate for DNA Synthesis (Material)

Deoxyribonucleoside triphosphate or a deoxyribonucleoside triphosphate derivative is used as a substrate for DNA synthesis. A substrate corresponding to template DNA in use is prepared. For example, when activated double-stranded DNA is used as the template DNA, as a general rule, four types of substrates, which are deoxyadenosine triphosphate (dATP) or a derivative thereof, deoxycytidine triphosphate (dCTP) or a derivative thereof, deoxyguanosine triphosphate (dGTP) or a derivative thereof, and thymidine triphosphate (dTTP) or a derivative thereof, are used in combination. On the other hand, when a polynucleoside chain constituted with one kind of deoxyribonucleoside and a primer complementary to the polynucleoside chain are used as template DNA, as a general rule, one kind of deoxyribonucleoside triphosphate or a derivative thereof, which is in concert with the polynucleoside chain used as the template, is used. For example, when polyadenylic acid and an oligo (dT) primer are employed, thymidine triphosphate (dTTP) or a derivative thereof is used for the substrate.

The “derivative” herein is not particularly limited as long as it is used as a substrate of a DNA polymerase in the same manner as general deoxyribonucleoside triphosphate and induces synthesis and elongation of a double-stranded DNA chain. Deoxyribonucleoside triphosphate formed into a derivative, which is obtained by labeling with a radioactive isotope (such as ³H and ³²P) or a fluorescence substance (such as Cy™ 3, Cy™ 5, Texas Red™ and fluorescein), introduction of a protective group, or substitution of a specific atom group, can be used as the “deoxyribonucleoside triphosphate derivative”.

In one embodiment of the present invention, labeling is simultaneously performed in the synthesis of a double-stranded DNA chain (that is, in the step (1)). For example, using deoxyribonucleoside triphosphate labeled with a radioactive isotope (e.g., ³H, and ³²P) or a fluorescence substance (e.g., Cy™ 3, Cy™ 5, Texas Red™, and fluorescein) as one substrate, labeled double-stranded DNA is synthesized by letting the double-stranded DNA incorporate the labeled deoxyribonucleoside triphosphate. In the case of this embodiment, double-stranded DNA is detected by use of the incorporated label.

The condition for incubation in the step (1) is not particularly limited as long as a mtDNA polymerase shows an activity and double-stranded DNA in a detectable level is synthesized. A person skilled in the art can set a suitable incubation condition through a preliminary experiment, or the like. An example of the incubation condition includes incubation at a temperature from 30° C. to 40° C. for 5 minutes to 6 hours. The incubation condition is preferably incubation at a temperature from 35° C. to 40° C. for 10 minutes to 3 hours, and more preferably incubation at a temperature of about 37° C. for 15 minutes to 1 hour.

Synthesized double-stranded DNA is detected in the step (2). In general, synthesized double-stranded DNA is detected after termination of the reaction. However, positive termination of the reaction is not essential. Detection with time or real-time detection may also be carried out. Since a mtDNA polymerase shows a bivalent iron ion requirement, for example, addition of a chelating agent is effective in order to terminate the reaction. However, the reaction may be terminated by another technique as long as the technique does not affect on detection of synthesized double-stranded DNA.

Various methods can be used in the detection herein. For example, two methods shown in examples described below, that is, a method using a radioactive isotope and a method using fluorescence can be employed. In the case of the former method, a radioactive isotope-labeled deoxyribonucleoside triphosphate is incorporated during the synthesis of double-stranded DNA (that is, step (1)), the intake amount of the radioactive isotope is measured by a liquid scintillation counter, or the like. On the other hand, in the method using fluorescence, a fluorescence amount is measured with a fluorescence reader, or the like, for example, by adding a fluorescence substance specific to double-stranded DNA and staining the synthesized double-stranded DNA with fluorescence. Examples of the fluorescence substance specific to double-stranded DNA include PicoGreen™, and SYBR Green I™. Note that fluorescence-labeled double-stranded DNA may also be synthesized by letting the double-stranded DNA incorporate fluorescence-labeled deoxyribonucleoside triphosphate during the synthesis of the double-stranded DNA (that is, the step (1)). That is, fluorescence labeling may also be performed at the same time as the synthesis of the double-stranded DNA.

By the way, there is a fear that bivalent iron in a reaction solution becomes trivalent iron by oxidation and precipitation of Fe(OH₃) is thus generated in the step (1) although it depends on incubation conditions and measurement conditions. Formation of the precipitation causes quenching, which is an obstacle to the measurement. Thus, in order to avoid such a problem, gallic acid, or the like are added to the reaction solution and trivalent iron is preferably formed into a complex.

The activity of the mtDNA polymerase is calculated in the step (3), using the detection result in the step (2). Typically, the amount of the mtDNA polymerase activity is quantified from the detected value, and semiquantitative or qualitative determination may also be employed.

In one embodiment of the present invention, incubation in the step (1) is carried out in the presence of a test substance, an influence of the test substance given to the activity of the mtDNA polymerase is determined based on an activity value calculated in the step (3). In other words, an action and effect of the test substance on the mtDNA polymerase activity are evaluated. For example, when decrease of the mtDNA polymerase activity is observed by addition of the test substance, it can be determined that the test substance has an inhibition action to the mtDNA polymerase. On the contrary, when increase of the mtDNA polymerase activity is observed by addition of the test substance, it can be determined that the test substance has a promoting action of the activity of the mtDNA polymerase. Thus, the activity measurement method of the present invention is useful for evaluating an action and effect of a test substance on the mtDNA polymerase activity. As one utilization form of the activity measurement method of the present invention, a screening method focusing on this respect will be described below.

General conditions for a DNA polymerase reaction may be adopted for the other conditions that are not particularly mentioned in the above explanation (such as other components and reaction conditions). With respect to this point, for example, Molecular Cloning (Third Edition, Cold Spring Harbor Laboratory Press, New York), Current protocols in molecular biology (edited by Frederick M. Ausubel et al., 1987), and the like can be referred. Note that a use amount of the mtDNA polymerase, a use amount of template DNA, a use amount of a substrate in the reaction solution in the step (1), and the like can be set in consideration of an intended use and other conditions of the activity measurement method. A person skilled in the art can determine suitable use amounts of respective materials by referring general reaction conditions of a DNA polymerase and past reports, or performing preliminary experiments. Examples of use amounts are shown below.

mtDNA polymerase: 5 μg/ml to 50 μg/ml

Template DNA: 50 μg/ml to 5000 μg/ml (in the case of using activated DNA), 0.1 nM to 10 nM (in the case of using a polynucleoside chain and a complementary primer)

Substrate: 1 mM to 1.6 mM (total amount)

### 2. Method for Screening for Anti-Malaria Compound

The second aspect of the present invention relates to a method for screening for an anti-malaria compound. A compound selected by the method for screening of the present invention is a promising active ingredient or leading compound of an anti-malaria agent. The following steps (i) to (iii) are carried out in the method for screening of the present invention:

(i) incubating a solution containing a bivalent iron ion, a mitochondrial DNA polymerase of falciparum malaria, template DNA, and at least one deoxyribonucleoside triphosphate or deoxyribonucleoside triphosphate derivative in the presence of a test substance;

(ii) detecting the synthesized double-stranded DNA; and,

(iii) determining effectiveness of the test substance based on the result of the detection carried out in step (ii), wherein inhibition of double-stranded DNA synthesis is indicative of effectiveness.

Since the steps (i) and (ii) are respectively the same as the steps (1) and (2) of the activity measurement method of the present invention except for using a test substance, explanation of the details about the steps (i) and (ii) are omitted. Organic compounds or inorganic compounds having various molecular sizes can be used for the test substance. Examples of the organic compounds include nucleic acid, peptide, protein, lipid (simple lipid, complex lipid (such as phosphoglyceride, sphingolipid, glycosyl glyceride, and cerebroside), prostaglandin, isoprenoid, terpene, steroid, polyphenol, catechin, and vitamins (such as B1, B2, B3, B5, B6, B7, B9, B12, C, A, D, and E). A test substance may be a substance derived from a natural product or a substance obtained by synthesis. In the case of the latter, for example, a technique of combinatorial synthesis is utilized and an effective screening system can be established. Note that a plant extraction, a cell extraction, a culture supernatant, or the like may be used as a test substance. In addition, an existing medical agent may be used as a test substance. A reciprocal action, a synergistic action, etc. among test substances may be examined by simultaneously adding two or more test substances.

Effectiveness of a test substance is determined in the step (iii) based on the detection result of the step (ii). An effective test substance is thus selected based on the determination result. In the present invention, “inhibition of double-stranded DNA synthesis” is adopted as an indication of effectiveness of the test substance. That is, when inhibition of double-stranded DNA synthesis is observed, a test substance is determined to be effective, and when inhibition of double-stranded DNA synthesis is not observed, a test substance is determined to be not effective. When a plurality of test substances are used, effectiveness of each test substance can be evaluated in comparison based on degrees of inhibition.

In general, a control group, which is incubated in the absence of a test substance (other conditions are the same as those in the step (i)), is prepared as a comparative object, and detection in the control group (the step (ii)) is carried out in parallel. Then, the detection result of the control group and the detection result of the test group are compared to each other to thus determine whether the test substance inhibits synthesis of double-stranded DNA or not. When effectiveness of the test substance is determined by comparison to the control group as described above, more credible determination result is obtained. The numbers of samples in the test group and the control group are not particularly limited. In general, as the number of samples in use is larger, a more credible result is obtained, but handling the large number of samples at the same time causes difficulty mainly in operation. Thus, the number of samples contained in each group is, for example, 1 to 50, preferably 2 to 30, and more preferably 3 to 20.

On the test substance which showed effectiveness in the step (iii), presence or absence and/or a degree of an inhibition activity to a nuclear DNA polymerase (DNA polymerase α, DNA polymerase β, etc.) of a malaria parasite may be evaluated. When it is revealed that the inhibition activity is not shown to the nuclear DNA polymerase as a result of this additional step, the test substance can be evaluated as a substance having high specificity to the mtDNA polymerase. A test substance evaluated as described above can be a promising active ingredient or leading compound of an anti-malaria agent closely targeting to mtDNA. On the other hand, a test substance found out to show an inhibition activity also to the nuclear DNA polymerase can be expected to have medicinal benefits targeting to the nuclear DNA polymerase as well as the mtDNA polymerase. Therefore, beneficial information for development and practical application of an anti-malaria agent can be obtained by addition of the above described step.

On the other hand, in order to evaluate the toxicity thereof, the test substance which showed effectiveness in the step (iii) may be confirmed that an inhibition activity to a human DNA polymerase is not shown.

When a substance selected by the method for screening of the present invention has sufficient medicinal benefits, the substance can be directly used as an active ingredient of an anti-malaria agent. On the other hand, when a substance does not have sufficient medicinal benefits, the substance may be subjected to alternation such as chemical modification to enhance its medicinal benefits and can be then used as an active ingredient of an anti-malaria agent. Needless to say, even when the substance has sufficient medicinal benefits, similar alternation may be performed for the purpose of further increase of medicinal benefits.

### EXAMPLES

Characteristics of the mitochondrial DNA polymerase were specifically examined aiming for clarification of a mitochondrial DNA replication mechanism of a malaria parasite.

### 1. Methods

(1) Full-Length Cloning of PFF1225c

The cell strain 3D7 of falciparum malaria was cultured in a human erythrocyte by following the reported method (Trager W and Jensen J B, Science. 1976 Aug. 20; 193(4254): 673-675), which was added with partial change. The malaria parasite in the trophozoite stage was recovered and the total RNA was extracted using RNeasy (QIAGEN). Then, cDNA was prepared using GeneRacer™ (Invitrogen). The full length sequence of PFF1225c (4335 bp, ACCESSION (GenBank) XM_{—}961143, DEFINITION Plasmodium falciparum 3D7 DNA polymerase 1, putative (PFF1225c) mRNA, complete cds.) (SEQ ID No. 8) was cloned using the 3′ race and 5′ race methods.

### (2) Expression and Purification of Recombinant Protein Using Wheat Germ Cell-Free Expression System

Wheat germ cell-free expression was carried out using ENDEXT (registered trademark) Wheat Germ Expression H Kit manufactured by CellFree Sciences Co., Ltd.

### (2-1) Preparation of Expression Vector

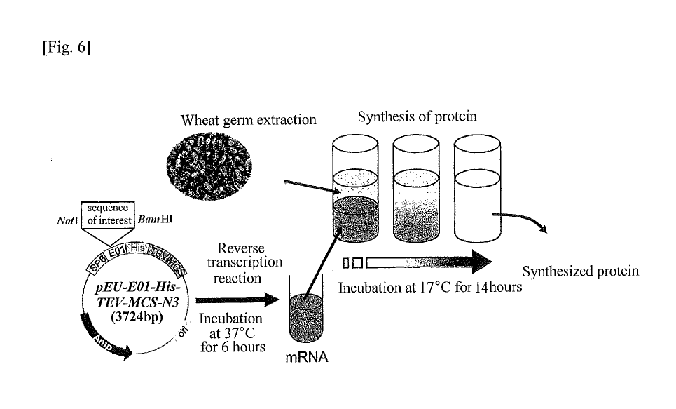

Partial sequences having various lengths, which contain a DNA polymerase domain (pol Ac) of PFF1225c (A1: 104-1444 a.a (SEQ ID No. 2), A2: 276-1444 a.a (SEQ ID No. 3), B1: 426-1444 a.a (SEQ ID No. 4), B2: 618-1444 a.a (SEQ ID No. 5), C1: 732-1444 a.a (SEQ ID No. 6), and C2: 990-1444 a.a (SEQ ID No. 7)) were inserted in frame to the BamHI/HindIII site of the vector (pEU-E01-His-TEV-MCS—N3: FIG. 6) in the wheat germ cell-free expression system. Bacteria in 600 ml of the LB liquid medium were collected to purify a plasmid using a QIAGEN plasmid Plus Midi kit (QIAGEN). In this time, purification was carried out without adding RNase attached to the kit. Then, the purified plasmid was precipitated with propanol and suspended in TE.

### (2-2) Transcription Reaction

120 μg of the purified vector, 240 μl of a 5× transcription buffer, 120 μl of 25 mM NTPs, 15 μl of a 80 U/μ1 RNase inhibitor, and 15 μl of 80 U/μl SP6 polymerase were mixed and adjusted to be a total of 1200 μl with milliQ water, and the reaction solution was incubated in a water bath at 37° C. for 6 hours. The reaction solution was stood still at room temperature after completion of the reaction.

### (2-3) Translation Reaction

The translation reaction was carried out with a 6-well plate. SUB-AMIX (registered trademark) (translation substrate) of the upper layer liquid was separately injected in each amount of 4.4 ml into one well, and then, 400.4 μl of the lower layer liquid was still overlapped so that the liquid surface was not disrupted. The lower layer liquid was mixed with 200 μl of mRNA, 0.4 μl of 40 mg/ml creatine kinase, and 200 μl of WEG (wheat germ extraction). The reaction solution was sealed with a lid for the purpose of prevention of drying and incubated in an incubator at 17° C. for 16 hours. After completion of the synthesis, the whole amount was transferred to a tube by gently pipetting.

### (2-4) His-Tag Purification

Imidazole (pH 8.0) was added to the total protein (4.8 ml), which was the translation reaction product, at a concentration of 20 mM and well mixed. The supernatant was separately obtained after centrifuging at 8000 rpm and 4° C. for 20 minutes, 50 μl of Ni-beads (Ni-NTA Superflow, QIAGEN) was added to the supernatant, and the mixture was gently stirred for 16 hours. Thereafter centrifugation was carried out at 4000 rpm and 4° C. for 5 minutes to remove the supernatant and Ni-beads were washed twice with 0.5 ml of a washing buffer (20 mM phosphoric acid buffer, 30 mM imidazole, 300 mM NaCl), and finally eluted with 50 μl of an elution buffer (20 mM phosphoric acid buffer, 500 mM imidazole, 300 mM NaCl) three times. Each eluted fraction was dialyzed with a dialysis buffer (50 mM Tris-HCl (pH 7.5), 10% glycerin, 1 mM EDTA, 5 mM mercaptoethanol, 0.1% NP-40) for 12 hours to remove imidazole.

(3) DNA Polymerase Activity Measurement using RI

A reaction solution was obtained by adding 50 mM Tris-HCl, 0.5 mM dATP, dGTP, dCTP, 5 μM dTTP, activated DNA (0.5 μg/ml), 0.8 μM [³H]dTTP (Moravek Co.: (MT-781) Thymidine 5′-triphosphate, tetrasodium salt, [methyl ³H]), and an enzyme solution was further added thereto, a metallic ion, an inhibitor, etc. having respective concentrations (the total amount of 10 μl). 0.1 μg of a C1 fragment was added per 10 μl. The mixed reaction solution was incubated at 37° C. for 30 minutes, then adsorbed to filter paper, dried, and washed with 5% Na₂HPO₄ 4 times (for 10 minutes for each washing), thereafter washing twice with distilled water (DW) (for 5 minutes for each washing), and finally shaken with 100% ethanol for 5 minutes. The filter paper was then dried and the dried filter paper was contained in a vial container charged with 4 ml of a toluene cocktail, and an intake amount of ³H was measured with a liquid scintillation counter.

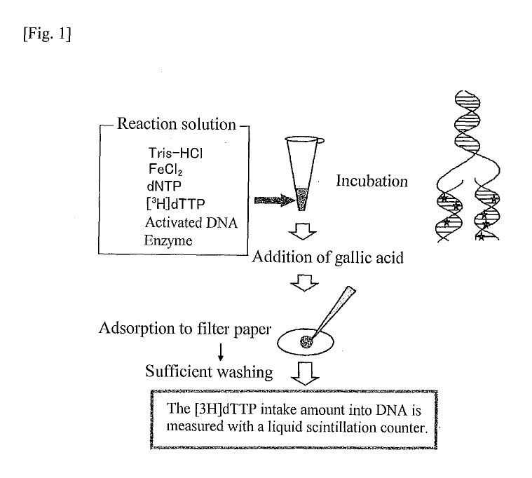

(4) DNA Polymerase Activity Measurement using RI in the Case of Adding Iron Ion (FIG. 1)

When a ³H amount taken in a DNA chain is measured with a liquid scintillation counter, the pigment of an iron ion causes quenching and an accurate measurement thus cannot be attained. Quenching is classified into three types such as chemical quenching, oxygen quenching, and coloration quenching, and calculation efficiency of a liquid scintillation counter decreases. The case of ³H being a soft β ray particularly becomes a problem, and coloration quenching becomes a problem in the case of an iron ion. Thus, gallic acid that forms a complex with iron was used and gallic acid iron that is a purple complex was formed. As a result, quenching was able to be significantly reduced. The protocol in the case of adding an iron ion is shown below.

The reaction solution was obtained by adding 50 mM Tris-HCl, 0.5 mM dATP, dGTP, dCTP, 5 μM dTTP, activated DNA (0.5 μg/ml), 0.8 μM [³H]dTTP (Moravek Co.: (MT-781) Thymidine 5′-triphosphate, tetrasodium salt, [methyl ³H]), and 10 mM FeCl₂ to be the total amount of 9 μl. In order to prevent oxidation of iron, the reaction solution was exposed to a nitrogen gas for 30 minutes to be degassed. 1 μl (0.1 μg) of a C1 fragment was added per 9 μl of the reaction solution. The mixed reaction solution was incubated at 37° C. for 30 minutes, and 10 μl of 1% gallic acid, which was the equal amount to the reaction solution, was then added and well suspended to form gallic acid iron. Then, the whole amount was adsorbed to filter paper, dried, and washed with 5% Na₂HPO₄ 10 times (totally for 30 minutes), thereafter washing twice with distilled water (DW) (for 5 minutes for each washing), and finally shaken with 100% ethanol for 5 minutes. The filter paper was then dried and the dried filter paper was contained in a vial container charged with 4 ml of a toluene cocktail, and an intake amount of ³H was measured with a liquid scintillation counter.

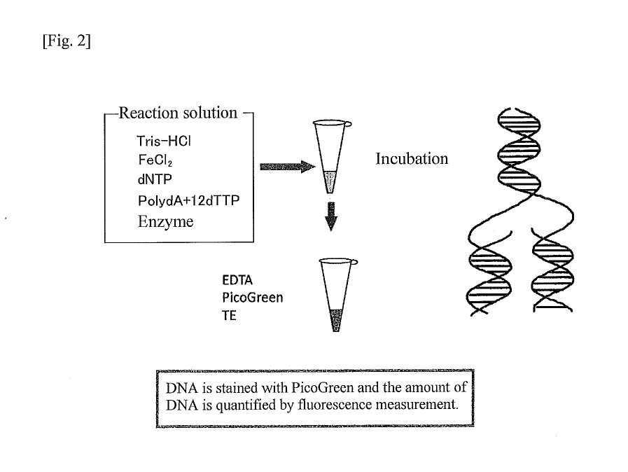

(5) DNA Polymerase Activity Measurement using PicoGreen™ (FIG. 2)

The reaction solution obtained by adding 50 mM Tris-HCl (pH 7.5), 1 mM dTTP, 40 nM PolydA-dT12, and 10 mM FeCl₂ and adjusted to be the total amount of 20 μl with degassed distilled water (DW). 5 μl of a C1 fragment (0.2 μg protein/μl) was added thereto, and the reaction solution was incubated at 37° C. for 30 minutes. After the incubation, an iron ion was chelated by adding 10 μl of 100 mM EDTA to terminate the reaction. PicoGreen™ (Molecular Probes), which specifically binds to double-stranded DNA, was used in order to quantitatively determine an amount of DNA synthesized by the DNA polymerase. 200 μl of PicoGreen™, which was diluted by 1/200 with TE, and 10 μl of the above described reaction product were mixed on a 96-well plate and CytoFluor™ Multi Well Plate Reader series 4000 (Applied Biosystems) was used to measure the DNA amount under the conditions of an excitation wavelength (Ex): 485/20 and a fluorescence wavelength (Em): 530/25.

### 2. Results

### (1) Identification of Mitochondrial DNA Polymerase of Malaria Parasite

A mitochondrial DNA polymerase found so far in animals is only DNA polymerase y (pol γ). However, homologs of pol γ have not been found in plants and algae. In recent years, DNA polymerases analogous to the DNA polymerase I (pol I) of E. coli were identified from rice, arabidopsis, tobacco, and red algae, which are higher plants (Christensen et al. Plant Cell.17(10): 2805-2816 2005, Kimura et al. Nucleic Acids Res. 1; 30(7): 1585-1592 2002, Mori et al. Biochem Biophys Res Commun. 19; 334(1): 43-50. 2005, Ono et al. Plant Cell Physiol. 48(12): 1679-1692. 2007). It was found that these enzymes locally exist in both of plastids and mitochondria and have enzymatic activities. In additon, a mitochondrial DNA polymerase (PpPolA) analogous to the DNA polymerase I was also identified from Physarum polycephalum, which is a protozoa, in our research laboratory. PpPoIA is the most analogius to the DNA polymerase I and was expected to be a primitive mitochondrial DNA polymerase.

Thus, BLAST search was performed with PlasmoDB that is a databse of a malaria parasite, using the PpPolA sequence. As a result, PF14_{—}0112, PFF1225c, PFB0180w were obtained as sequences with high homology. When the domain structures of these sequneces were estimated in a plurality of sites such as PROSITE, sequneces having the DNA polymerase domain were two sequneces of PF14_{—}0112 and PFF1225c. One of them, PF14_{—}0112 has been already reported as the apicoplast DNA polymerase (Seow et al. Molecular & Biochemical Parasitology 141: 145-153 2005). On the other hand, PFF1225c was annotated as a DNA polymerase I-like protein, but the functions thereof, and the like have not been analyzed yet. As a result of analyzing by use of PlasMit that is an intracellular local existence prediction site of a malaria parasite, local existence in mitochondria was predicted. Furthermore, also from an analysis actually using GFP, local existence in mitochondria was shown. Therefore, PFF1225c was expected to have high possibility to function as a mitochondrial DNA polymerase.

### (2) Expression of PFF1225c Recombinant Protein Using Wheat Germ Cell-Free Expression System

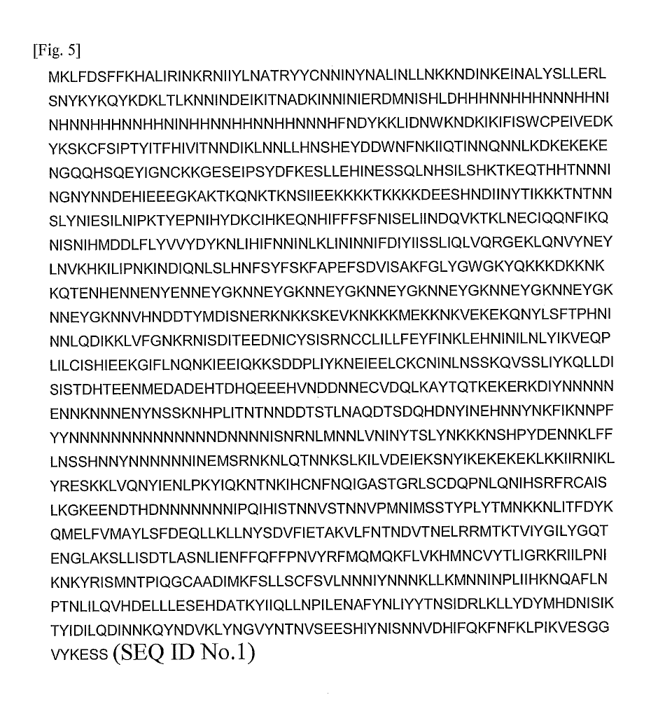

The full-length of PFF1225c was cloned using cDNA of falciparum malaria and, as a result, found to be a protein made of 1444 amino acids (FIG. 5). In addition, it was also found by homology search that PFF1225c was a protein analogous to the DNA polymerase I, which contains a DNA polymerase domain (polAc) in the C terminal side (1126-1335a.a).

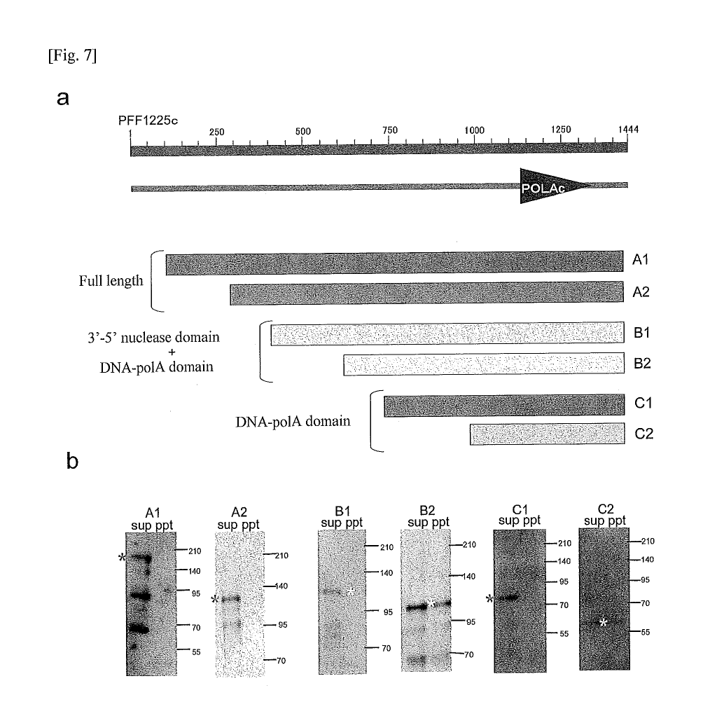

In order to confirm whether PFF1225c actually has the DNA polymerase activity or not, a PFF1225c recombinant protein was tried to be expressed using E. coli. However, the PFF1225c recombinant protein could not to be expressed in the E. coli system. Thus, expression was tried using a wheat germ cell-free expression system, which has been reported to efficiently express a protein of a malaria parasite (FIG. 6). Partial sequences with various lengths containing a DNA polymerase domain (polAc) of PFF1225c (A1: 104-1444 a.a (SEQ ID No. 2), A2: 276-1444 a.a (SEQ ID No. 3), B1: 426-1444 a.a (SEQ ID No. 4), B2: 618-1444 a.a (SEQ ID No. 5), C1: 732-1444 a.a (SEQ ID No. 6), and C2: 990-1444 a.a (SEQ ID No. 7)) were expressed in the wheat germ cell-free expression system; as a result, all recombinant proteins were confirmed to have been expressed in solubilized fractions (FIG. 7). In particular, the expression amount of C1 was higher and C1 was thus used in analyses in the following.

### (3) DNA Polymerase Activity Measurement Using Protein Prepared in Wheat Germ Cell-Free Expression System

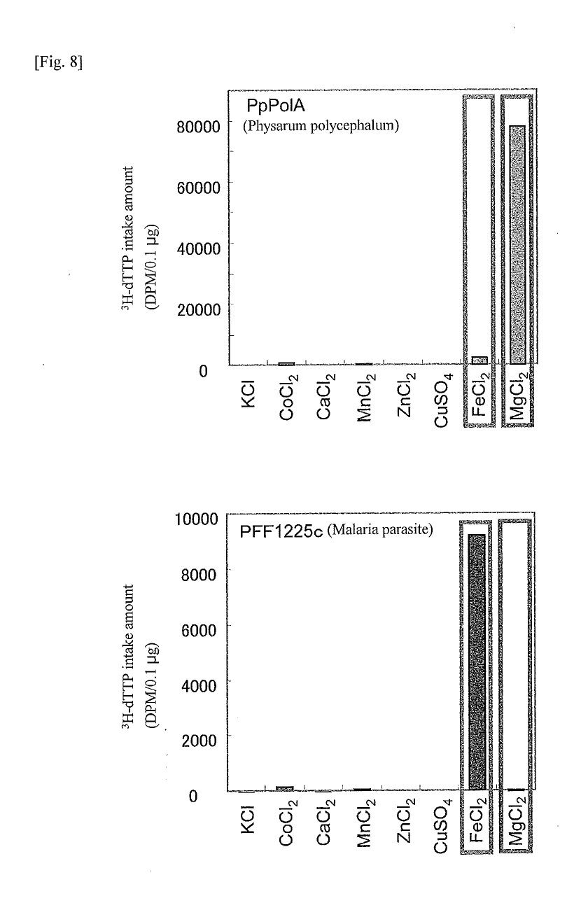

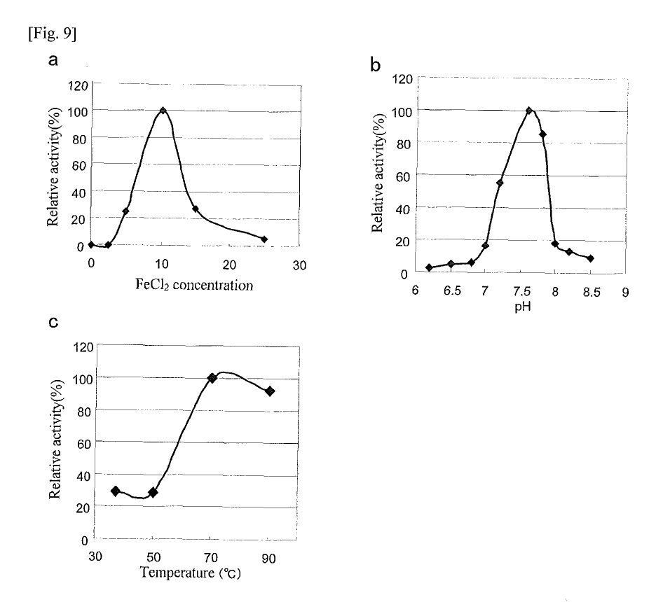

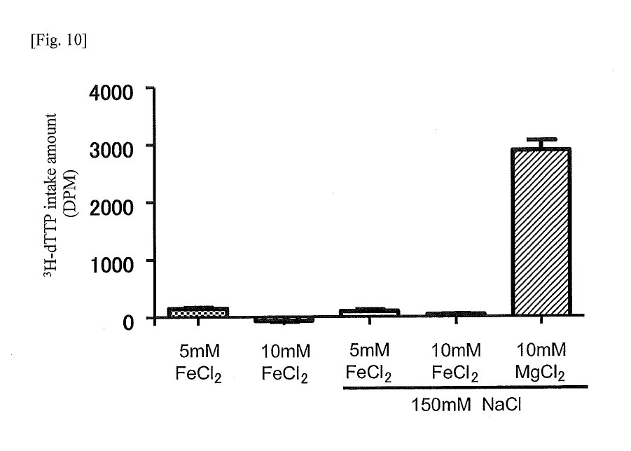

A DNA polymerase generally requires bivalent metallic ions such as magnesium and manganese for its activity. Firstly, in order to clarify a metallic ion requirement of PFF1225c, an enzyme activity was measured with an intake of [³H]dTTP using various bivalent metallic ions (10 mM). In a mitochondrial DNA polymerase (PpPolA) in Physarum polycephalum, a high activity was shown only at the time of adding a Mg²⁺ ion, as generally reported, but no activity was shown in PFF1225c with addition of a Mg²⁺ ion, and a high activity was only shown when a Fe²⁺ ion was added (FIG. 8). In addition, the highest activity was shown when a bivalent iron ion concentration was 10 mM (FIG. 9a). The activation of a DNA polymerase by addition of a Fe²⁺ ion as described above was not observed also when an assay was carried out using a human mitochondrial DNA polymerase γ (FIG. 10), and therefore, can be a sepecific property to the mitochondrial DNA polymerase of a malaria parasite. Furthermore, as a result of study on an optimum pH, the highest activity was shown at pH 7.5 (FIG. 9b).

Next, in order to examine heat resistance of an enzyme, an enzyme solution was previously pre-incubated at each temperature for 5 minutes and then cooled with ice, thereafter carrying out a usual measurement of a DNA polymerase activity. As a result, very interestingly, the enzyme was not deactivated even by the thermal treatment at 90° C., adversely, the activity was increased by the thermal treatment and the highest activity was shown by the thermal treatment at 70° C. (about 3 times higher activity in comparison to the case of the treatment at 37° C.) (FIG. 9c).

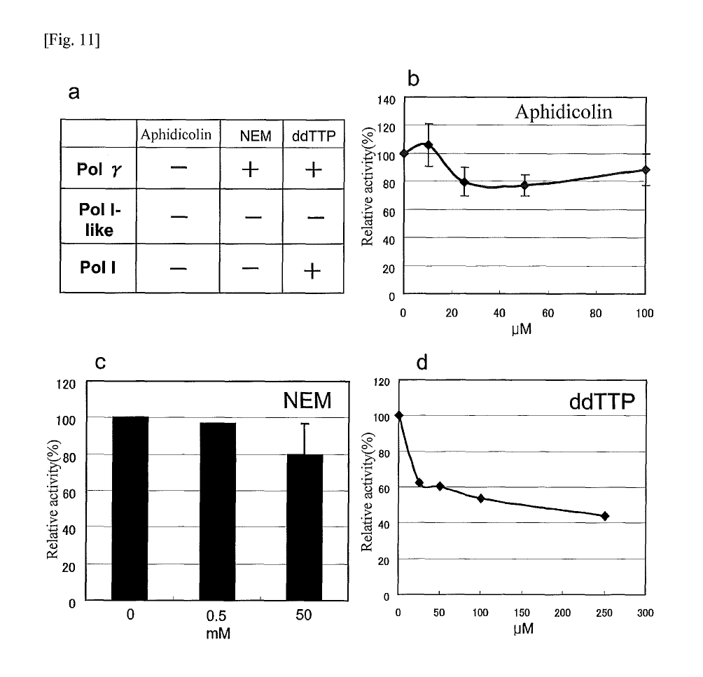

Furthermore, sensitivity to an inhibitor of a DNA polymerase was studied (FIG. 11). Firstly, when aphidicolin that is a specific inhibitor of a DNA polymerase α of a cell nucleus was added at a concentration from 10 to 100 μM, the activity scarcely decreased (FIG. 11b). In addition, when NEM and ddTTP, which are inhibitors to the DNA polymerase γ, were also examined on sensitivity thereof, in the case of NEM, the activity scarcely decreased even in the presence at a high concentration (FIG. 11c). It was reported that, in the case of ddTTP, the activity thereof is mostly inhibited at a concentration from 50 to 100 mM in the DNA polymerase γ, and in the case of PFF1125c, although a weak inhibition effect was shown along with increasing a concentration, the activity was reduced only to 50% even when the concentration was increased to 400 mM (FIG. 11d). It was found that such sensitivity to an inhibitor was similar to that of a mitochondrial DNA polymerase of myxomycetes and plants, which have been reported so far, and different from the DNA polymerase γ.

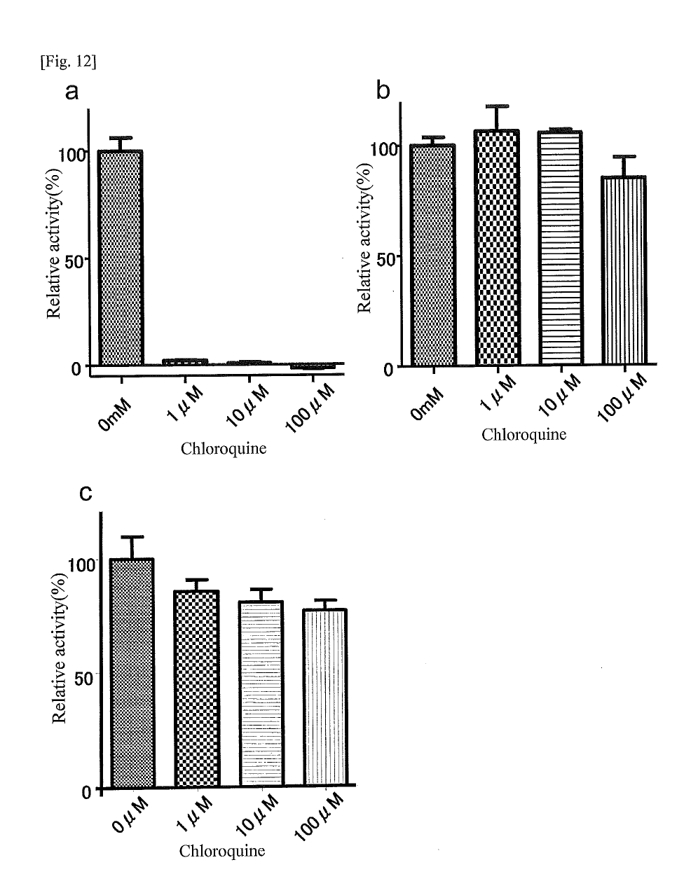

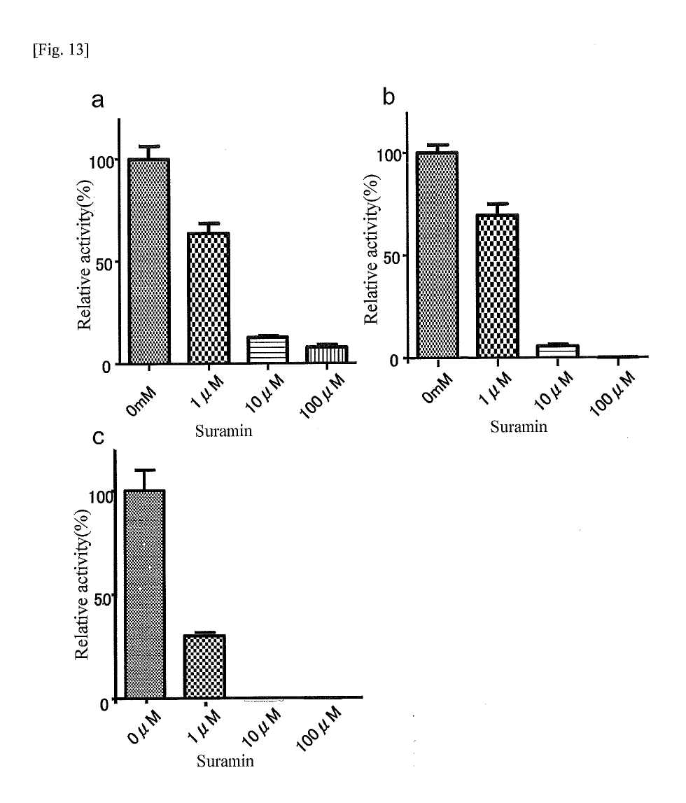

Next, sensitivity to chloroquine and suramin, which were reported as inhibitors of the apicoplast DNA polymerase (POMUPfprex) of a malaria parasite, were examined. Chloroquinea and suramin were used as treatment agents for malaria and trypanosoma, respectively, and it has been already reported that chloroquine does not inhibit activities of the mouse DNA polymerase α and the DNA polymerase γ. Firstly, when an inhibition action to the DNA polymerase activity of chloroquine was examined, an inhibition effect was not shown in the human DNA polymerase γ and PpPolA of Physarum polycephalum, but chloroquine inhibited the activity of PFF1225c (FIG. 12). Furthermore, it was reported that suramin binds non-specifically to various DNA polymerases and inhibits the activities thereof, and suramin also inhibited the activity of PFF1225c (FIG. 13).

### (4) Measurement of DNA Polymerase Activity of Malaria Parasite Mitochondrial Crude Fraction

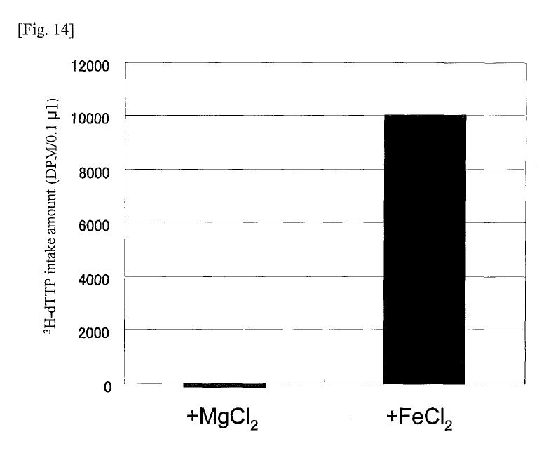

It was revealed from the above described analysis that PFF1225c prepared in the wheat germ cell-free expression system does not show the DNA synthesis activity in the presence of Mg²⁺ ion but exerts the activity by addition of Fe²⁺ ion. However, Charavalitshewinkoon-Permitr, et al. reported in 2000 that PFF1225c has the DNA synthesis activity in the presence of Mg²⁺ ion in an analysis using a mitochondrial fraction obtained by crude purification from a malaria parasite (Charavalitshewinkoon-Permits et al., Parasitology International 49 279-288 2000). It can be considered that apicoplast was mixed in the mitochondrial fraction in the analysis and there is a possibility that the activity of the apicoplast DNA polymerase, which shows a Mg²⁺ ion requirement, was measured. Thus, a mitochondrial fraction without mixing apicoplast was prepared and the DNA polymerase activity was measured. As a result, the DNA synthesis activity was not shown in the presence of Mg²⁺ ion also in the mitochondrial fraction, and it was found that the activity was shown only at the time of addition of Fe²⁺ ion (FIG. 14).

### (5) Optimization of Codon

C1 showing the highest expression amount was expressed using a DNA sequence (SEQ ID No. 10) in which a codon was optimized. As a result, increase of about 1.5 to 2 times of an expression amount was observed (no data is shown). Note that when the E. coli expression system was used, significant improvement in production efficiency was observed due to optimization of a codon (the optimized DNA sequence was set forth in SEQ ID No. 9) (no data is shown).

### 3. Conclusion

As described above, expression of a mitochondrial DNA polymerase of a malaria parasite was succeeded by using a cell-free expression system and, at the same time, it was clarified that the DNA polymerase shows a Fe²⁺ requirement. In addition, useful and important findings for the measurement for the activity of the DNA polymerase such as concentration dependency and pH dependency regarding Fe²⁺ were obtained.

### INDUSTRIAL APPLICABILITY

The activity measurement method of the present invention is useful in, for example, searching a compound showing an anti-malaria activity. Furthermore, the invention is also useful as a research tool for a mtDNA polymerase of a malaria parasite.

The invention is not construed by the description of embodiments and examples of the invention described above at all. Various modified embodiments are also included in the invention within the range that a person skilled in the art can easily conceive of, without deviating from the description of the scope of patent claims.

Contents of treatises, unexamined patent publications, and examined patent publications specified in this specification are all incorporated herewith by their references.

1. A method for measuring the activity of a DNA polymerase, comprising the following steps of (1) to (3):
(1) incubating a solution containing a bivalent iron ion, a mitochondrial DNA polymerase of falciparum malaria, template DNA, and at least one deoxyribonucleoside triphosphate or deoxyribonucleoside triphosphate derivative;
(2) detecting the synthesized double-stranded DNA; and
(3) calculating the activity of the DNA polymerase from the result of the detection carried out in step (2).

2. The method for measuring the activity of a DNA polymerase according to claim 1, wherein the mitochondrial DNA polymerase includes any one of sequences set forth in SEQ ID NOs. 1 to 7 or partially altered sequences thereof and shows the DNA polymerase activity.

3. The method for measuring the activity of a DNA polymerase according to claim 1, wherein the mitochondrial DNA polymerase is a protein prepared in a cell-free synthesis system.

4. The method for measuring the activity of a DNA polymerase according to claim 1, wherein the template DNA is activated double-stranded DNA, or a combination of one-stranded DNA or a polynucleotide chain constituted with one kind of deoxyribonucleotide, and a complementary primer thereof.

5. The method for measuring the activity of a DNA polymerase according to claim 1, wherein the detection of the double-stranded DNA is carried out by fluorescence staining specific to double-stranded DNA.

6. The method for measuring the activity of a DNA polymerase according to claim 1, wherein the concentration of the bivalent iron ion in the solution is 5 mM to 15 mM.

7. The method for measuring the activity of a DNA polymerase according to claim 1, wherein the pH of the solution is 7 to 8.

8. The method for measuring the activity of a DNA polymerase according to claim 1, wherein the mitochondrial DNA polymerase is thermally pretreated in the temperature condition from 50° C. to 90° C.

9. The method for measuring the activity of a DNA polymerase according to claim 1, wherein the incubation in the step (1) is carried out in the presence of a test substance.

10. A method for screening for an anti-malaria compound comprising the following steps of (i) to (iii):
(i) incubating a solution containing a bivalent iron ion, a mitochondrial DNA polymerase of falciparum malaria, template DNA, and at least one deoxyribonucleoside triphosphate or deoxyribonucleoside triphosphate derivative in the presence of a test substance;
(ii) detecting the synthesized double-stranded DNA; and,
(iii) determining effectiveness of the test substance based on the result of the detection carried out in step (ii), wherein inhibition of double-stranded DNA synthesis is indicative of effectiveness.

11. The screening method according to claim 10, wherein a sample (control group) incubated under the same conditions as in the step (i) except for the absence of a test substance is prepared and effectiveness in the step (iii) is determined by comparing to the detection result for the control group in the step (ii).

12. The screening method according to claim 10, further comprising a step of evaluating an inhibition activity to a nuclear DNA polymerase of falciparum malaria for a test substance which shows effectiveness in the step (iii).

13. The screening method according to claim 10, further comprising a step of confirming that a test substance which shows effectiveness in the step (iii) shows no inhibition activity to a human DNA polymerase.
